(19) United States
(12) Patent Application Publication
(10) Pub. No.: US 2023/0265053 A1
(43) Pub. Date: Aug. 24, 2023

(54) POLYFUNCTIONAL QUATERNARY AMMONIUM SALT OF SULFONIC ACID HAVING ACTIVE-HYDROGEN GROUPS

(71) Applicant: TOKUYAMA CORPORATION, Shunan-shi, Yamaguchi (JP)

(72) Inventors: Yasutomo SHIMIZU, Tsukuba-shi (JP); Takayoshi KAWASAKI, Tsukuba-shi (JP)

(73) Assignee: TOKUYAMA CORPORATION, Shunan-shi, Yamaguchi (JP)

(21) Appl. No.: 17/927,182

(22) PCT Filed: May 27, 2021
(86) PCT No.: PCT/JP2021/020277

§ 371 Date: Nov. 22, 2022

(30) Foreign Application Priority Data
May 28, 2020 (JP) 2020-093008

(51) Int. Cl. C07C 309/18; C07D 233/58; C08G 18/38; C08G 18/08; C08G 18/10; B24B 37/24

(52) U.S. Cl. CPC C07C 309/18; B24B 37/24; C07D 233/58; C08G 18/10; C08G 18/0828; C08G 18/3857

(57) ABSTRACT

The polyfunctional active hydrogen group-containing sulfonate quaternary ammonium salt of the present invention is a polyfunctional active hydrogen group-containing sulfonate quaternary ammonium salt having at least two active hydrogen groups in a molecule, and the polyfunctional active hydrogen group-containing sulfonate quaternary ammonium salt has a quaternary ammonium salt skeleton of the polyfunctional active hydrogen group-containing sulfonate quaternary ammonium salt that includes an imidazole skeleton. According to the present invention, a polyfunctional active hydrogen group-containing sulfonate quaternary ammonium salt that is excellent in solubility and dispersibility in a polyurethane (urea) resin raw material, and a high effect of the hydrophilization of a polyurethane (urea) resin can be provided.

### TECHNICAL FIELD

The present invention relates to a polyfunctional active hydrogen group-containing sulfonate quaternary ammonium salt. More specifically, the present invention relates to a polyfunctional active hydrogen group-containing sulfonate quaternary ammonium salt having at least two active hydrogen groups in the molecule and an imidazole skeleton as a quaternary ammonium salt skeleton.

### BACKGROUND ART

A compound having an ionic group has been applied to various purposes as materials for a surfactant, a medical intermediate, an organic chemical intermediate, and an ionic liquid, and for hydrophilization of a polyurethane, a polyester, and the like, and has been used, for example, in automobile interior and exterior, aqueous inks, adhesives, various coating agents, and the like.

In recent years, it has been known that a hydrophilic group, such as an ionic group, is introduced to a polyurethane (urea) resin constituting a polishing pad for chemical mechanical polishing (CMP) used for wafer polishing, thereby providing a polishing pad for CMP having a large polishing rate and is hard to cause scratches on the surface of the wafer as a polishing object (PTL 1).

As the hydrophilization of a polyurethane (urea) resin in this manner, a method of introducing an ionic group, such as an anionic group or a cationic group, to a polyurethane (urea) resin has been known, and various methods have been known for the hydrophilization of the resin, for example, by introducing an ionic group, such as a sulfonic acid group, a carboxylic acid group, and a quaternary ammonium group, to one component constituting the resin.

Specifically, the known methods of introducing the ionic group to the polyurethane (urea) resin include a method of using a raw material monomer or oligomer having the ionic group, and in the case where the ionic group is introduced to the polyurethane (urea) resin, in general, a method of using a diol component having the ionic group as the raw material monomer or oligomer is employed, and for example, a method of using a diol component containing a sulfonic acid or a carboxylic acid (or a salt thereof) has been known.

As the carboxylic acid group-containing diol, 3,3-dimethylolpropionic acid and the like have been known, but it has been known that a carboxylic acid group is insufficient in effect of hydrophilization due to the weak anionicity thereof.

On the other hand, the sulfonate salt diol component generally has low solubility in the low boiling point solvent and the raw material monomer or oligomer used for producing the polyurethane (urea) resin, and is difficult to use particularly in the production of the polyurethane (urea) resin with no solvent. For example, PTL 1 describes the production of a polyurethane (urea) resin using N,N-bis(2-hydroxyethyl)-2-aminoethanesulfonic acid, but the content of N,N-bis(2-hydroxyethyl)-2-aminoethanesulfonic acid in the polyurethane (urea) resin is low due to the low solubility of the N,N-bis(2-hydroxyethyl)-2-aminoethanesulfonic acid in the polyurethane (urea) resin raw materials, and thus is difficult to exhibit the sufficient effect. In response to this, a method of enhancing the solubility by making N,N-bis(2-hydroxyethylaminoethanesulfonic acid to a quaternary ammonium salt (PTL 2). However, the resulting compound in the method of PTL 2 is still in a solid state at room temperature, and the further enhancement of the solubility has been demanded for using as a polyurethane (urea) resin raw material.

### CITATION LIST

### Patent Literatures

PTL 1: JP 2007-276061 A
PTL 2: JP 2001-354742 A

### SUMMARY OF INVENTION

### Technical Problem

Accordingly, an object of the present invention is to provide a polyfunctional active hydrogen group-containing sulfonate quaternary ammonium salt that is excellent in solubility and dispersibility in a polyurethane (urea) resin raw material, and has a high effect of the hydrophilization of a polyurethane (urea) resin. In particular, an object thereof is to provide a polyfunctional active hydrogen group-containing sulfonate quaternary ammonium salt that can be favorably used as one component constituting a polishing pad for CMP.

### Solution to Problem

The present inventors have been made earnest investigations for solving the problem. As a result, it has been found that in the case where a polyfunctional active hydrogen group-containing sulfonate quaternary ammonium salt is obtained by using a polyfunctional active hydrogen group-containing sulfonic acid and a quaternary ammonium salt including an imidazole skeleton, not only an excellent solubility in a monomer or an oligomer as a polyurethane (urea) resin raw material can be obtained, but also the hydrophilicity of the polyurethane (urea) resin can be enhanced by incorporating the polyfunctional active hydrogen group-containing sulfonate quaternary ammonium salt to the polyurethane (urea) resin through reaction with an iso(thio)cyanate, and thus the present invention has been completed. The present invention relates to the novel polyfunctional active hydrogen group-containing sulfonate quaternary ammonium salt.

Specifically, the present invention relates to a polyfunctional active hydrogen group-containing sulfonate quaternary ammonium salt having at least two active hydrogen groups in a molecule and an imidazole skeleton as a quaternary ammonium salt skeleton.

The present invention also relates to a polishing pad for CMP including the polyfunctional active hydrogen group-containing sulfonate quaternary ammonium salt.

### Advantageous Effects of Invention

The polyfunctional active hydrogen group-containing sulfonate quaternary ammonium salt of the present invention has a feature that the polyfunctional active hydrogen group-containing sulfonate quaternary ammonium salt has at least two active hydrogen groups in the molecule, and the quaternary ammonium salt skeleton of the polyfunctional active hydrogen group-containing sulfonate quaternary ammonium salt includes an imidazole skeleton. According to the feature, excellent solubility in a monomer or an oligomer as a polyurethane (urea) resin raw material can be obtained. The production of a polyurethane (urea) resin containing the polyfunctional active hydrogen group-containing sulfonate quaternary ammonium salt can impart excellent hydrophilicity to the resin. For example, the use of the polyurethane (urea) resin as a polishing pad for CMP can enhance the interaction with the slurry and can exhibit excellent polishing characteristics. For example, a high polishing rate and the reduction of defects occurring in wafers can be achieved.

Furthermore, the polyfunctional active hydrogen group-containing sulfonate quaternary ammonium salt of the present invention can be applied to various other fields than the polishing pad for CMP, examples of which include the fields of water absorption requiring absorption and retention of water, for example, the field of a hygiene material, such as a sanitary product and a disposable diaper, the field of an excrement treatment material for animals, the field of agriculture and horticulture, the field of foods, such as freshness keeping, and the industrial field, such as dew condensation prevention and a refrigerant, and also include interior and exterior materials of automobiles, aqueous inks, adhesives, and various coating agents.

### DESCRIPTION OF EMBODIMENTS

### Polyfunctional Active Hydrogen Group-Containing Sulfonate Quaternary Ammonium Salt

The polyfunctional active hydrogen group-containing sulfonate quaternary ammonium salt of the present invention (which may be hereinafter referred to as a component (A)) may be any of a polyfunctional active hydrogen group-containing sulfonate quaternary ammonium salt that has at least two active hydrogen groups in the molecule, and has a quaternary ammonium salt skeleton of the polyfunctional active hydrogen group-containing sulfonate quaternary ammonium salt that includes an imidazole skeleton, with no limitation.

In particular, the polyfunctional active hydrogen group-containing sulfonate quaternary ammonium salt that is in a liquid state and has fluidity at room temperature is preferred. According to the configuration, dissolution or mixing thereof in a monomer or an oligomer as a polyurethane (urea) resin raw material can be achieved.

In the description herein, the room temperature means 25° C.

In particular, the polyfunctional active hydrogen group-containing sulfonate quaternary ammonium salt of the present invention is preferably a polyfunctional active hydrogen group-containing sulfonate quaternary ammonium salt represented by the following formula (1).

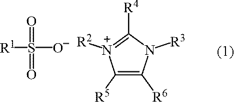

In the formula, R¹ represents an organic group having at least two active hydrogen groups.

In the formula R² and R³ each independently represent a linear alkyl group having 1 to 20 carbon atoms, a linear alkenyl group having 1 to 20 carbon atoms, a branched alkyl group having 3 to 20 carbon atoms or a branched alkenyl group having 3 to 20 carbon atoms, in which a part of the linear alkyl group, the linear alkenyl group, the branched alkyl group, or the branched alkenyl group may be substituted by an —O— bond, a —CO— bond, a —COO— bond, an —NH— bond, an —SO—bond, or an —SiO— bond, a part of the hydrogen atoms of the linear alkyl group, the linear alkenyl group, the branched alkyl group, or the branched alkenyl group may be substituted by at least one kind selected from the group consisting of a hydroxy group and a phenyl group, and the groups represented by R² and R³ may be the same as or different from each other.

In the formula, R⁴ represents a methyl group or a hydrogen atom.

In the formula, R⁵ and R⁶ each independently represent a hydrogen atom, a linear alkyl group having 1 to 20 carbon atoms, a linear alkenyl group having 1 to 20 carbon atoms, a branched alkyl group having 3 to 20 carbon atoms or a branched alkenyl group having 3 to 20 carbon atoms, in which a part of the linear alkyl group, the linear alkenyl group, the branched alkyl group, or the branched alkenyl group may be substituted by an —O— bond, a —CO— bond, a —COO— bond, an —NH— bond, an —SO— bond, or an —SiO— bond, a part of the hydrogen atoms of the linear alkyl group, the linear alkenyl group, the branched alkyl group or the branched alkenyl group may be substituted by at least one kind selected from the group consisting of a hydroxy group and a phenyl group, the groups represented by R⁵ and R⁶ may be the same as or different from each other, and R⁵ and R⁶ and the carbon atoms bonded thereto may form an aliphatic hydrocarbon ring having 3 to 20 ring carbon atoms.

A more preferred embodiment of the polyfunctional active hydrogen group-containing sulfonate quaternary ammonium salt of the present invention is a polyfunctional active hydrogen group-containing sulfonate quaternary ammonium salt represented by the following formula (2).

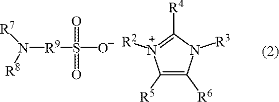

In the formula, R², R³, R⁴, R⁵, and R⁶ have the same meanings as in the formula (1).

In the formula, R⁷ and R⁸ each independently represent a hydrogen atom, a linear alkyl group having 2 to 20 carbon atoms, at least one hydrogen atom of which is substituted by an active hydrogen group, or a branched alkyl group having 3 to 20 carbon atoms, at least one hydrogen atom of which is substituted by an active hydrogen group, in which a part of the linear alkyl group or the branched alkyl group may be substituted by an —O— bond, a —CO— bond, a —COO— bond, or an —NH— bond, a part of the hydrogen atoms other than the active hydrogen group of the linear alkyl group or the branched alkyl group may be substituted by a phenyl group, and the groups represented by R⁷ and R⁸ may be the same as or different from each other, but do not represent hydrogen atoms simultaneously.

In the formula, R⁹ represents a linear alkylene group having 1 to 20 carbon atoms or a branched alkylene group having 3 to 20 carbon atoms, in which a part of the linear alkylene group or the branched alkylene group may be substituted by an —O— bond, a —CO— bond, a —COO— bond, or an —NH— bond, and a part of the hydrogen atoms of the linear alkylene group or the branched alkylene group may be substituted by at least one kind selected from the group consisting of an active hydrogen group and a phenyl group.

In the formula, R⁷, R⁸, and R⁹ have at least two active hydrogen groups in total.

The active hydrogen group in R¹ in the formula (1) is preferably at least one kind of a group selected from the group consisting of a hydroxy group, a thiol group, and an amino group, more preferably at least one kind of a group selected from the group consisting of a hydroxy group and an amino group, and further preferably a hydroxy group.

In particular, R² in the formulae (1) and (2) further preferably represents a linear or branched alkyl group having 1 to 4 carbon atoms.

Furthermore, R³ in the formulae (1) and (2) further preferably represents a linear or branched alkyl group having 1 to 20 carbon atoms, in which a part of the linear alkyl group or the branched alkyl group may be substituted by an —O—bond, a —CO— bond, a —COO— bond, or an —NH— bond, and a part of the hydrogen atoms of the linear alkyl group or the branched alkyl group may be substituted by at least one kind selected from the group consisting of a hydroxy group and a phenyl group.

In particular, R³ in the formulae (1) and (2) preferably represents a linear or branched alkyl group having 1 to 20 carbon atoms, in which a part of the linear alkyl group or the branched alkyl group may be substituted by a —CO— bond or a —COO— bond, and a part of the hydrogen atoms of the linear alkyl group or the branched alkyl group may be substituted by at least one kind selected from the group consisting of a hydroxy group and a phenyl group. In the present invention, the water absorption rate can be controlled by changing the length of R³. In a cured product using the polyfunctional active hydrogen group-containing sulfonate quaternary ammonium salt of the present invention, particularly for such purposes as a polishing pad for CMP, R³ preferably represents a linear or branched alkyl group having 3 to 20 carbon atoms. In the case where the number of carbon atoms is in the range, excellent mechanical characteristics can be exhibited. In the various purposes of water absorption, for example, the field of a hygiene material, such as a sanitary product and a disposable diaper, the field of an excrement treatment material for animals, the field of agriculture and horticulture, the field of foods, such as freshness keeping, and the industrial field, such as dew condensation prevention and a refrigerant, R³ preferably represents a linear or branched alkyl group having 1 to 4 carbon atoms.

R⁴ in the formulae (1) and (2) further preferably represents a hydrogen atom.

R⁵ and R⁶ in the formulae (1) and (2) further preferably represent hydrogen atoms.

It is further preferred that R⁷ and R⁸ in the formula (2) each independently represent a group represented by the following formula (3) or (4), or R⁷ represents a hydrogen atom or a methyl group, and R⁸ represents a group represented by any one of the following formulae (5) to (7). In the case where R⁷ represents a hydrogen atom or a methyl group, and R⁸ represents a group represented by the following formula (7), R⁹ contains at least one active hydrogen group.

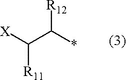

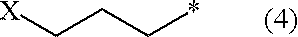

In the formulae, X represents a hydroxy group or an amino group. In particular, X preferably represents a hydroxy group.

In the formulae, R¹¹ and R¹² each independently represent a linear or branched alkyl group having 1 to 10 carbon atoms, or a phenyl group, in which a part of the alkyl group may be substituted by an —O— bond or an —NH— bond. In particular, both R¹¹ and R¹² preferably represent hydrogen atoms. In the formulae, * represents a bonding site bonded to the nitrogen atom.

In particular, both R⁷ and R⁸ in the formula (2) preferably represent groups represented by the formula (3).

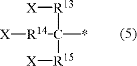

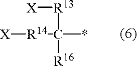

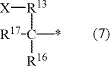

In the formulae, X has the same meaning as in the formulae (3) and (4).

In the formulae, R¹³, R¹⁴, and R¹⁵ each independently represent a linear alkylene group having 1 to 3 carbon atoms, or the alkylene group, a part of which may be substituted by a hydroxy group.

In the formulae, R¹⁶ and R¹⁷ each independently represent a hydrogen atom, a methyl group, or an ethyl group. In the formulae, * represents a bonding site bonded to the nitrogen atom.

R⁹ in the formulae (1) and (2) further preferably represents a linear alkylene group having 1 to 10 carbon atoms, in which a part of the linear alkylene group may be substituted by an —O— bond, and a part of the hydrogen atoms of the linear alkylene group may be substituted by at least line kind selected from the group consisting of a hydroxy group and an amino group.

### Production Method of Polyfunctional Active Hydrogen Group-Containing Sulfonate Quaternary Ammonium Salt

The production method of the polyfunctional active hydrogen group-containing sulfonate quaternary ammonium salt of the present invention is not limited, and for example, the polyfunctional active hydrogen group-containing sulfonate quaternary ammonium salt can be produced through reaction of a corresponding polyfunctional active hydrogen group-containing sulfonic acid or a corresponding polyfunctional active hydrogen group-containing sulfonate salt with a corresponding imidazolium salt.

In particular, the production is preferably performed by an ion exchange method. As a specific example, the polyfunctional active hydrogen group-containing sulfonate quaternary ammonium salt of the present invention can be produced in such a manner that the anion component of the imidazolium salt is exchanged to a hydroxide ion with an ion exchange resin, and then the imidazolium salt is mixed with the polyfunctional active hydrogen group-containing sulfonic acid (or a salt thereof), followed by distilling off water. By exchanging the imidazolium salt to a hydroxide ion in advance as described above, only water is formed in the production of the polyfunctional active hydrogen group-containing sulfonate quaternary ammonium salt of the present invention by mixing with the polyfunctional active hydrogen group-containing sulfonic acid, examples of the advantages of which include easiness in the post-treatment.

The polyfunctional active hydrogen group-containing sulfonic acid or sulfonate salt used may be a known compound with no limitation, and for example, may be produced by the method described in JP 2010-163376 A, in which taurine and an oxirane compound are reacted to form a sulfonate salt.

Specific examples thereof include N,N-bis(2-hydroxyethyl)-2-aminoethanesulfonic acid, N,N-bis(2-hydroxyethyl)-3-aminopropanesulfonic acid, N,N-bis(2-hydroxyethyl)-3-amino-2-hydroxypropanesulfonic acid, N,N-bis(2-hydroxypropyl)-3-aminopropanesulfonic acid, N-tris(hydroxymethyl)methyl-2-aminoethanesulfonic acid, N-tris(hydroxymethyl)methyl-2-aminopropanesulfonic acid, 3-[N-tris(hydroxymethyl)methylamino]-2-hydroxypropanesulfonic acid, and inorganic salts thereof, but the present invention is not limited to these examples.

The imidazolium salt may be any imidazolium salt that is formed of an imidazolium cation component and an anion component with no particular limitation.

Specific examples of the imidazolium cation component include 1,3-dimethylimidazolium cation, 1,3-diethylimidazolium cation, 1-ethyl-3-methylimidazolium cation, 1-propyl-3-methylimidazolium cation, 1-butyl-3-methylimidazolium cation, 1-hexyl-3-methylimidazolium cation, 1-methyl-3-octylimidazolium cation, 1-decyl-3-methylimidazolium cation, 1-dodecyl-3-methylimidazolium cation, 1-tetradecyl-3-methylimidazolium cation, 1-pentadecyl-3-methylimidazolium cation, 1-hexadecyl-3-methylimidazolium cation, 1-octadecyl-3-methylimidazolium cation, 1-allyl-3-methylimidazolium cation, 3-ethyl-1-vinylimidazolium cation, 1-(2-hydroxyethyl)-3-methylimidazolium cation, 1-benzyl-3-methylimidazolium cation, 1-ethyl-2,3-dimethylimidazolium cation, 1-butyl-2,3-dimethylimidazolium cation, 1,2-dimethyl-3-propylimidazolium cation, 1-hexyl-2,3-dimethylimidazolium cation, 1,2,3-trimethylimidazolium cation,1,2,3,4-tetramethylimidazolium, 1,3,4-trimethylimidazolium cation, 2-ethyl-1,3,4-trimethylimidazolium cation, 1,3-dimethyl-2,4-diethylimidazolium cation, 1,2-dimethyl-3,4-diethylimidazolium cation, 1-methyl-2,3,4-triethylimidazolium cation, 1,2,3,4-tetraethylimidazolium cation, 1,3-dimethyl-2-ethylimidazolium cation, 1-ethyl-2,3-dimethylimidazolium cation, 1,2,3-triethylimidazolium cation, 1-butyl-3,4,5-trimethylimidazolium cation, 1-butyl-2,3,4,5-tetramethylimidazolium cation, 1,1-dimethylimidazolium cation, 1,1,2-trimethylimidazolium, 1,1,2,4-tetramethylimidazolium, and 1,1,2,5-tetramethylimidazolium, but the present invention is not limited to these examples.

In the polyfunctional active hydrogen group-containing sulfonate quaternary ammonium salt that is particularly preferred in the present invention, the polyfunctional active hydrogen group-containing sulfonic acid is selected from N,N-bis(2-hydroxyethyl)-2-aminoethanesulfonic acid, N,N-bis(2-hydroxyethyl)-3-aminopropanesulfonic acid, and N,N-bis(2-hydroxyethyl)-3-amino-2-hydroxypropanesulfonic acid, and the cation component of the imidazolium salt is selected from 1,3-dimethylimidazolium cation, 1-ethyl-3-methylimidazolium cation, 1-propyl-3-methylimidazolium cation, 1-butyl-3-methylimidazolium cation, 1-hexyl-3-methylimidazolium cation, 1-methyl-3-octylimidazolium cation, 1-decyl-3-methylimidazolium cation, 1-dodecyl-3-methylimidazolium cation, 1-tetradecyl-3-methylimidazolium cation, 1-pentadecyl-3-methylimidazolium cation, 1-hexadecyl-3-methylimidazolium cation, 1-octadecyl-3-methylimidazolium cation, 1-ethyl-2,3-dimethylimidazolium cation, 1-butyl-2,3-dimethylimidazolium cation, 1,2-dimethyl-3-propylimidazolium cation, 1-hexyl-2,3-dimethylimidazolium cation, and 1,2,3-trimethylimidazolium cation.

### Curable Composition

A curable composition of the present invention is a curable composition that contains the polyfunctional active hydrogen group-containing sulfonate quaternary ammonium salt (A) described above and a polymerizable monomer (B) having a polymerizable functional group capable of being polymerized with the polyfunctional active hydrogen group-containing sulfonate quaternary ammonium salt (A). As described later, a cured product obtained by curing the curable composition can be used, for example, as a polishing pad for CMP.

### Polymerizable Monomer Having Polymerizable Functional Group Capable of Being Polymerized With Polyfunctional Active Hydrogen Group-Containing Sulfonate Quaternary Ammonium Salt

In the present invention, the polymerizable monomer (B) having a polymerizable functional group capable of being polymerized with the polyfunctional active hydrogen group-containing sulfonate quaternary ammonium salt (A) (which may be hereinafter referred to as a component (B) or simply to as a polymerizable monomer) may be any known compound that is a polymerizable monomer having a group capable of being polymerized with the polyfunctional active hydrogen group of the polyfunctional active hydrogen group-containing sulfonate quaternary ammonium salt, and is naturally a compound other than the polyfunctional active hydrogen group-containing sulfonate quaternary ammonium salt.

Examples of the component (B) include a polyfunctional isocyanate compound having at least two isocyanate groups (B1) (which may be hereinafter referred to as a component (B1)), a polyfunctional isothiocyanate compound having at least two isothiocyanate groups (B2) (which may be hereinafter referred to as a component (B2)), an epoxy group-containing compound (B3) (which may be hereinafter referred to as a component (B3)), and an episulfide group-containing compound (B4) (which may be hereinafter referred to as a component (B4)). The polymerizable monomer having a polymerizable functional group capable of being polymerized with the polyfunctional active hydrogen group-containing sulfonate quaternary ammonium salt may be used alone or as a combination of two or more kinds thereof.

The component (B) is preferably the polyfunctional isocyanate compound having at least two isocyanate groups (B1) and/or the polyfunctional isothiocyanate compound having at least two isothiocyanate groups (B2), and most preferably the polyfunctional isocyanate compound having at least two isocyanate groups (B1). Excellent resin characteristics can be exhibited by polymerizing the component (B) described above and the polyfunctional active hydrogen group-containing sulfonate quaternary ammonium salt.

### Polyfunctional Isocyanate Compound Having at Least Two Isocyanate Groups (B1)

The component (B1) used in the present invention may be any polyfunctional isocyanate compound that has at least two isocyanate groups, with no limitation. In particular, a compound having 2 to 6 isocyanate groups in the molecule is preferred, and a compound having 2 or 3 isocyanate groups therein is more preferred.

The component (B1) may be a urethane prepolymer having isocyanate groups at ends thereof (B12) prepared through reaction of a bifunctional isocyanate compound and a bifunctional active hydrogen-containing compound described later (which may be hereinafter referred to as a urethane prepolymer (B12) or referred to as a component (B12)). The urethane prepolymer (B12) may be any one that contains isocyanate groups at ends thereof, with no limitation.

The component (B1) can be roughly classified into an aliphatic isocyanate, an alicyclic isocyanate, an aromatic isocyanate, other isocyanates, and the urethane prepolymer (B12). The component (B1) used may be a single compound or multiple kinds of the compounds. In the case where multiple kinds of the compounds are used, the mass thereof for standard is the total amount of the multiple kinds of the compounds. Specific examples of the isocyanate compounds include the following compounds.

### Aliphatic Isocyanate

Bifunctional isocyanate compounds (corresponding to the bifunctional isocyanate compound constituting the urethane prepolymer), for example, ethylene diisocyanate, trimethylene diisocyanate, tetramethylene diisocyanate, hexamethylene diisocyanate, octamethylene diisocyanate, nonamethylene diisocyanate, 2,2′-dimethylpentane diisocyanate, 2,2,4-trimethylhexamethylene diisocyanate, decamethylene diisocyanate, butene diisocyanate, 1,3-butadiene 1,4-diisocyanate, 2,4,4-trimethylhexamethylene diisocyanate, 1,6,11-trimethylundecamethylene diisocyanate, 1,3,6-trimethylhexamethylene diisocyanate, 1,8-diisocyanato-4-isocyanatomethyloctane, 2,5,7-trimethyl-1,8-diisocyanato-5-isocyanatomethyloctane, bis(isocyanatoethyl) carbonate, bis(isocyanatoethyl) ether, 1,4-butylene glycol dipropyl ether-ω,ω′-diisocyanate, lysine diisocyanatomethyl ester, and 2,4,4-trimethylhexamethylene diisocyanate.

### Alicyclic Isocyanate

Bifunctional isocyanate compounds (corresponding to the bifunctional isocyanate compound constituting the urethane prepolymer), for example, isophorone diisocyanate, (bicyclo[2.2.1]heptan-2,5-diyl)bismethylene diisocyanate, (bicyclo[2.2.1]heptan-2,6-diyl)bismethylene diisocyanate, 2B,5a-bis(isocyanato)norbornane, 2B,5B-bis(isocyanato)norbornane, 2B,6a-bis(isocyanato)norbornane, 2B,6B-bis(isocyanato)norbornane, 2,6-di(isocyanatomethyl)furan, 1,3-bis(isocyanatomethyl)cyclohexane, dicyclohexylmethane-4,4′-diisocyanate, 4,4-isopropylidene bis(cyclohexylisocyanate), cyclohexane diisocyanate, methylcyclohexane diisocyanate, dicyclohexyldimethylmethane diisocyanate, 2,2′-dimethyldicyclohexylmethane diisocyanate, bis(4-isocyanato-n-butylidene)pentaerythritol, dimer acid diisocyanate, 2,5-bis(isocyanatomethyl)-bicyclo[2-2-1]-heptane, 2,6-bis(isocyanatomethyl)-bicyclo[2-2-1]-heptane, 3,8-bis(isocyanatomethyl)tricyclodecane, 3,9-bis(isocyanatomethyl)tricyclodecane, 4,8-bis(isocyanatomethyl)tricyclodecane, 4,9-bis(isocyanatomethyl)tricyclodecane, 1,5-diisocyanatodecalin, 2,7-diisocyanatodecalin, 1,4-diisocyanatodecalin, 2,6-diisocyanatodecalin, bicyclo[4.3.0]nonane-3,7-diisocyanate, bicyclo[4.3.0]nonane-4,8-diisocyanate, bicyclo[2.2.1]heptane-2,5-diisocyanate and bicyclo[2.2.1]heptane-2,6-diisocyanate, bicyclo[2,2,2]octane-2, 5-diisocyanate, bicyclo[2,2,2]octane-2,6-diisocyanate, tricyclo[5.2.1.02.6]decane-3,8-diisocyanate, and tricyclo[5.2.1.02.6]decane-4,9-diisocyanate.

Polyfunctional isocyanate compounds, for example, 2-isocyanatomethyl-3-(3-isocyanatopropyl)-5-isocyanatomethyl-bicyclo[2,2,1]-heptane, 2-isocyanatomethyl-3-(3-isocyanatopropyl)-6-isocyanatomethyl-bicyclo[2,2,1]-heptane, 2-isocyanatomethyl-2-(3-isocyanatopropyl)-5-isocyanatomethyl-bicyclo[2,2,1]-heptane, 2-isocyanatomethyl-2-(3-isocyanatopropyl)-6-isocyanatomethyl-bicyclo[2,2,1]-heptane, 2-isocyanatomethyl-3-(3-isocyanatopropyl)-5-(2-isocyanatoethyl)-bicyclo[2,2,1]-heptane, 2-isocyanatomethyl-3-(3-isocyanatopropyl)-6-(2-isocyanatoethyl)-bicyclo[2,2,1]-heptane, 2-isocyanatomethyl-2-(3-isocyanatopropyl)-5-(2-isocyanatoethyl)-bicyclo[2,2,1]-heptane, 2-isocyanatomethyl-2-(3-isocyanatopropyl)-6-(2-isocyanatoethyl)-bicyclo[2,2,1]-heptane, and 1,3,5-tris(isocyanatomethyl)cyclohexane.

### Aromatic Isocyanate

Bifunctional isocyanate compounds (corresponding to the bifunctional isocyanate compound constituting the urethane prepolymer), for example, xylylene diisocyanate (o-, m-, or p-), tetrachloro-m-xylylene diisocyanate, methylene diphenyl-4,4′-diisocyanate, 4-chloro-m-xylylene diisocyanate, 4,5-dichloro-m-xylylene diisocyanate, 2,3,5,6-tetrabromo-p-xylylene diisocyanate, 4-methyl-m-xylylene diisocyanate, 4-ethyl-m-xylylene diisocyanate, bis(isocyanatoethyl)benzene, bis(isocyanatopropyl)benzene, 1,3-bis(a,a-dimethylisocyanatomethyl)benzene, 1,4-bis(a,a-dimethylisocyanatomethyl)benzene, a,a,a′,a′-tetramethylxylylene diisocyanate, bis(isocyanatobutyl)benzene, bis(isocyanatomethyl)naphthalene, bis(isocyanatomethyl) diphenyl ether, bis(isocyanatoethyl) phthalate, 2,6-di(isocyanatomethyl)furan, phenylene diisocyanate (o-, m-, or p-), ethylphenylene diisocyanate, isopropylphenylene diisocyanate, dimethylphenylene diisocyanate, diethylphenylene diisocyanate, diisopropylphenylene diisocyanate, trimethylbenzene triisocyanate, benzene triisocyanate, 1,3,5-triisocyanatomethylbenzene, 1,5-naphthalene diisocyanate, methylnaphthalene diisocyanate, biphenyl diisocyanate, 2,4-tolylene diisocyanate, 2,6-tolylene diisocyanate, 4,4′-diphenylmethane diisocyanate, 2,2′-diphenylmethane diisocyanate, 2,4′-diphenylmethane diisocyanate, 3,3′-dimethyldiphenylmethane 4,4′-diisocyanate, bibenzyl-4,4′-diisocyanate, bis(isocyanatophenyl)ethylene, 3,3′-dimethoxybiphenyl-4,4′-diisocyanate, phenylisocyanatomethyl isocyanate, phenylisocyanatoethyl isocyanate, tetrahydronaphthalene diisocyanate, hexahydrobenzene diisocyanate, hexahydrodiphenylmethane-4,4′-diisocyanate, diphenyl ether diisocyanate, ethylene glycol diphenyl ether diisocyanate, 1,3-propylene glycol diphenyl ether diisocyanate, benzophenone diisocyanate, diethylene glycol diphenyl ether diisocyanate, dibenzofuran diisocyanate, carbazole diisocyanate, ethylcarbazole diisocyanate, dichlorocarbazole diisocyanate, 2,4-tolylene diisocyanate, and 2,6-tolylene diisocyanate.

Polyfunctional isocyanate compounds, for example, mesitylene triisocyanate, triphenylmethane triisocyanate, polymeric MDI, naphthalene triisocyanate, diphenylmethane-2,4,4′-triisocyanate, 3-methyldiphenylmethane-4,4′,6-triisocyanate, and 4-methyl-diphenylmethane-2,3,4′,5,6-pentaisocyanate.

### Other Polyfunctional Isocyanate Compound Having at Least Two Isocyanate Groups

Examples of the other isocyanate compound include a polyfunctional isocyanate having a burette structure, an uretdione structure, or an isocyanurate structure formed of a diisocyanate compound, such as hexamethylene diisocyanate, 2,4-tolylene diisocyanate, 2,6-tolylene diisocyanate, or xylylene diisocyanate (o-, m-, or p-), as a major raw material (for example, JP 2004-534870 A describes a modification method of a burette structure, an uretdione structure, or an isocyanurate structure of an aliphatic polyisocyanate), and an adduct having polyfunctionality with a trifunctional or higher functional polyol, such as trimethylolpropane (see, for example, “Polyurethane Jushi Handbook” (Polyurethane Resin Handbook), edited by Keiji IWATA, Nikkan Kogyo Shimbun, Ltd. (1987)).

### Urethane Prepolymer (B12)

In the present invention, the urethane prepolymer (B12) is preferably obtained through reaction of a bifunctional isocyanate compound selected from the (B1) component (i.e., the compound described as the component (B1)) with at least one kind of a compound selected from the group consisting of a bifunctional polyol compound (C12) (which may be hereinafter referred to as a component (C12)), a bifunctional amine compound (C22) (which may be hereinafter referred to as a component (C22)), and a bifunctional polythiol compound (C32) (which may be hereinafter referred to as a component (C32)).

In the present invention, however, the urethane prepolymer (B12) necessarily contains isocyanate groups at both ends thereof. The production method of the urethane prepolymer (B12) containing isocyanate groups at both ends thereof may be a known method with no particular limitation, and examples thereof include a production method performed in such a manner that the molar number (n5) of the isocyanate groups in the bifunctional isocyanate compound and the molar number (n6) of the groups having an active hydrogen of the component (C12), the component (C22), and the component (C32) are in a range of 1 < (n5)/(n6) ≤ 2.3. In the case where two or more kinds of the bifunctional isocyanate compounds are used, the molar number (n5) of the isocyanate groups is the total molar number of the isocyanate groups of the bifunctional isocyanate compounds. In the case where two or more kinds of each of the component (C12), the component (C22), and the component (C32) are used, the molar number (n6) of the groups having an active hydrogen is the total molar number of the active hydrogens of the component (C12), the component (C22), and the component (C32).

In the present invention, even in the case where the active hydrogen is in a primary amino group, the primary amino group is calculated as one mol. This is because the reaction of the second amino group (—NH) of the primary amino group requires considerable energy (i.e., the second —NH is difficult to react even in a primary amino group), and therefore in the present invention, even in the case where a bifunctional active hydrogen-containing compound having a primary amino group is used, the primary amino group is calculated as one mol.

While not limiting, the urethane prepolymer (B12) preferably has an isocyanate equivalent (i.e., a value obtained by dividing the molecular weight of the urethane prepolymer (B12) by the number of isocyanate groups in one molecule) of 300 to 5,000, more preferably 350 to 3,000, and particularly preferably 400 to 2,000. The urethane prepolymer (B12) in the present invention is preferably a linear compound produced from the bifunctional isocyanate compound, and the component (C12), the component (C22), or the component (C32), and in this case, both ends thereof are isocyanate groups, and the number of isocyanate groups in one molecule is two.

The isocyanate equivalent of the urethane prepolymer (B12) can be quantitatively determined by measuring the isocyanate groups of the urethane prepolymer (B12) through the following back titration method according to JIS K7301. Firstly, the resulting urethane prepolymer (B12) is dissolved in a dried solvent. Subsequently, di-n-butylamine having a known concentration in an amount that is apparently excessive to the amount of the isocyanate groups of the urethane prepolymer (B12) is added to the dried solvent, so as to react the entire isocyanate groups of the urethane prepolymer (B12) wit di-n-butylamine. Subsequently, the unconsumed di-n-butylamine (i.e., di-n-butylamine that has not participated in the reaction) is titrated with an acid, so as to obtain the consumed amount of di-n-butylamine. The consumed amount of di-n-butylamine and the amount of the isocyanate groups of the urethane prepolymer (B12) are the same as each other, and therefore the isocyanate equivalent can be obtained therefrom. For example, in the case where the linear urethane prepolymer (B12) containing isocyanate groups is used, the number average molecular weight of the urethane prepolymer (B12) is twice the isocyanate equivalent. The molecular weight of the urethane prepolymer (B12) readily agree with the value measured by gel permeation chromatography (GPC). In the case where the urethane prepolymer (B12) and the bifunctional isocyanate compound are used in combination, the mixture thereof may be measured according to the method described above.

The isocyanate content (I) (molality (mol/kg)) of the urethane prepolymer (B12) and the urethane bond content (U) (molality (mol/kg)) existing in the urethane prepolymer (B12) preferably satisfy 1 ≤ (U)/(I) ≤ 10. This range is also applied to the case where the urethane prepolymer (B12) and the bifunctional isocyanate compound are used in combination.

The isocyanate content (I) (molality (mol/kg)) is a value obtained by multiplying the reciprocal of the isocyanate equivalent by 1,000. The urethane bond content (U) (molality (mol/kg)) existing in the urethane prepolymer (B12) can be obtained by the following manner for the theoretical value thereof. Specifically, assuming that the content of the isocyanate groups existing in the bifunctional isocyanate compound constituting the urethane prepolymer (B12) before the reaction is the total isocyanate content ((aI), molality (mol/kg)), the urethane bond content ((U), molality (mol/kg)) is a value obtained by subtracting the isocyanate content ((I), molality (mol/kg)) from the total isocyanate content ((aI), molality (mol/kg)) of the component (B1), i.e., (U) = (aI)-(I) is the urethane bond content (U) existing in the urethane prepolymer (B12).

In the production of the urethane prepolymer (B12), heating and addition of a urethanation catalyst may be performed depending on necessity. The urethanation catalyst used may be optionally selected appropriately, and specific examples thereof include urethanation catalysts described later.

Preferred examples of the component (B1) used in the present invention from the standpoint of the reactivity and the controllability include an alicyclic isocyanate, such as isophorone diisocyanate, 1,3-bis(isocyanatemethyl)cyclohexane, and (bicyclo[2.2.1]heptan-2,5(2,6)-diyl)bismethylene diisocyanate, an aromatic diisocyanate, such as 2,4-tolylene diisocyanate, 2,6-tolylene diisocyanate, 4,4′-diphenylmethane diisocyanate, xylylene diisocyanate (o-, m-, or p-), and 1,5-naphthalene diisocyanate, a polyfunctional isocyanate having a burette structure, an uretdione structure, or an isocyanurate structure formed of a diisocyanate compound, such as hexamethylene diisocyanate, 2,4-tolylene diisocyanate, 2,6-tolylene diisocyanate, or xylylene diisocyanate (o⁻, m⁻, or p⁻), as a major raw material, an adduct having polyfunctionality with a trifunctional or higher functional polyol, and the urethane prepolymer (B12).

Among these, the component (B1) containing a polyfunctional isocyanate compound comprising of the urethane prepolymer (B12) is most preferred.

### Polyfunctional Isothiocyanate Compound Having at Least Two Isothiocyanate Groups (B2)

The polyfunctional isothiocyanate compound (B2) used in the present invention may be any polyfunctional isothiocyanate compound that has at least two isothiocyanate groups, with no limitation. In particular, a compound having 2 to 6 isothiocyanate groups in the molecule is preferred, and a compound having 2 or 3 isothiocyanate groups therein is more preferred.

The component (B2) may be a prepolymer having isothiocyanate groups at ends thereof (B22) (which may be hereinafter referred to as an isothiocyanate group-containing prepolymer (B22) or a component (B22)) prepared through reaction of a bifunctional isothiocyanate compound with a bifunctional active hydrogen-containing compound, such as the component (C12), the component (C22), and the component (C32). The isothiocyanate group-containing prepolymer or a component (B22) may be any one that contains isothiocyanate groups at ends thereof, and can be produced in the similar manner as the component (B12).

As the component (B2), one kind of the compound may be used, or multiple kinds of the compounds may be used. In the case where multiple kinds of the compounds are used, the mass thereof for standard is the total amount of the multiple kinds of the compounds.

Specific examples of the isothiocyanate compounds include the following compounds.

p-Phenylene diisothiocyanate, xylylene 1,4-diisothiocyanate, ethylidyne diisothiocyanate, and the isothiocyanate group-containing prepolymer (B22).

### Epoxy Group-Containing Compound (B3)

The epoxy group-containing compound has an epoxy group as a polymerizable group in the molecule, and the epoxy compound is roughly classified into an aliphatic epoxy compound, an alicyclic epoxy compound, and an aromatic epoxy compound. For preferred specific examples thereof, reference may be made to WO 2015/068798.

### Episulfide Group-Containing Compound (B4)

The epoxy group-containing compound has an episulfide group as a polymerizable group in the molecule, and for specific examples of the episulfide compound, reference may be made to WO 2015/068798.

### Active Hydrogen-Containing Compound (C) Other Than Polyfunctional Active Hydrogen Group-Containing Sulfonate Quaternary Ammonium Salt

In the present invention, the curable composition may contain an active hydrogen-containing compound (C) other than the polyfunctional active hydrogen group-containing sulfonate quaternary ammonium salt (which may be hereinafter referred to as a component (C)), which is an active hydrogen-containing compound other than the polyfunctional active hydrogen group-containing sulfonate quaternary ammonium salt. The compound contained enables control of the resin characteristics of the resulting cured product, and a further excellent cured product can be produced. The component (C) can be roughly classified into a polyol compound (C1) (which may be hereinafter referred to as a component (C1)), a polyamine component (C2) (which may be hereinafter referred to as a component (C2)), a polythiol component (C3) (which may be hereinafter referred to as a component (C3)), a compound containing at least two kinds of active hydrogen groups (C4) (which may be hereinafter referred to as a component (C4)), and a polyrotaxane containing at least two active hydrogen groups (C5).

### Polyol Compound (C1)

The polyol compound (C1) used in the present invention may be any polyol compound that has two or more hydroxy groups in one molecule, with no limitation. The polyol compound can be roughly classified into an aliphatic alcohol, an alicyclic alcohol, an aromatic alcohol, a polyester polyol, a polyether polyol, a polycaprolactone polyol, a polycarbonate polyol, a polyacrylic polyol, and a castor oil based polyol.

Specific examples of the polyol compound (C1) include the following compounds.

### Aliphatic Alcohol

Bifunctional polyol compounds (corresponding to the bifunctional polyol compound (C12) constituting the urethane prepolymer (B12)), for example, ethylene glycol, diethylene glycol, propylene glycol, dipropylene glycol, butylene glycol, 1,5-dihydroxypentane, 1,6-dihydroxyhexane, 1,7-dihydroxyheptane, 1,8-dihydroxyoctane, 1,9-dihydroxynonane, 1,10-dihydroxydecane, 1,11-dihydroxyundecane, 1,12-dihydroxydodecane, neopentyl glycol, glyceryl monooleate, monoelaidin, polyethylene glycol, 3-methyl-1,5-dihydroxypentane, dihydroxyneopentyl, 2-ethyl-1,2-dihydroxyhexane, and 2-methyl-1,3-dihydroxypropane.

Polyfunctional polyol compounds, for example, glycerin, trimethylolethane, trimethylolpropane, ditrimethylolpropane, trimethylolpropane tripolyoxyethylene ether (such as TMP-30, TMP-60, and TMP-90, available from Nippon Nyukazai Co., Ltd.), butanetriol, 1,2-methylglucoside, pentaerythritol, dipentaerythritol, tripentaerythritol, sorbitol, erythritol, threitol, ribitol, arabinitol, xylitol, allitol, mannitol, dulcitol, iditol, glycol, inositol, hexanetriol, triglycerol, diglycerol, and triethylene glycol.

### Alicyclic Alcohol

Bifunctional polyol compounds (corresponding to the bifunctional polyol compound (C12) constituting the urethane prepolymer (B12)), for example, hydrogenated bisphenol A, cyclobutanediol, cyclopentanediol, cyclohexanediol, cycloheptanediol, cyclooctanediol, cyclohexanedimethanol, hydroxypropylcyclohexanol, tricyclo[5,2,1,02,6]decane-dimethanol, bicyclo[4,3,0]-nonanediol, dicyclohexanediol, tricyclo[5,3,1,13,9]dodecanediol, bicyclo[4,3,0]nonanedimethanol, tricyclo[5,3,1,13,9]dodecane-diethanol, hydroxypropyltricyclo[5,3,1,13,9]dodecanol, spiro[3,4]octanediol, butylcyclohexanediol, 1,1′-bicyclohexylidenediol, 1,4-cyclohexanedimethanol, 1,3-cyclohexanedimethanol, 1,2-cyclohexanedimethanol, and o-dihydroxyxylylene.

Polyfunctional polyol compounds, for example, tris(2-hydroxyethyl) isocyanurate, cyclohexanetriol, sucrose, maltitol, and lactitol.

### Aromatic Alcohol

Bifunctional polyol compounds (corresponding to the bifunctional polyol compound (C12) constituting the urethane prepolymer (B12)), for example, dihydroxynaphthalene, dihydroxybenzene, bisphenol A, bisphenol F, xylylene glycol, tetrabromobisphenol A, bis(4-hydroxyphenyl)methane, 1,1-bis(4-hydroxyphenyl)ethane, 1,2-bis(4-hydroxyphenyl)ethane, bis(4-hydroxyphenyl)phenylmethane, bis(4-hydroxyphenyl)diphenylmethane, bis(4-hydroxyphenyl)-1-naphthylmethane, 1,1-bis(4-hydroxyphenyl)-1-phenylethane, 2-(4-hydroxyphenyl)-2-(3-hydroxyphenyl)propane, 2,2-bis(4-hydroxyphenyl)butane, 1,1-bis(4-hydroxyphenyl)butane, 2,2-bis(4-hydroxyphenyl)-3-methylbutane, 2,2-bis(4-hydroxyphenyl)pentane, 3,3-bis(4-hydroxyphenyl)pentane, 2,2-bis(4-hydroxyphenyl)hexane, 2,2-bis(4-hydroxyphenyl)octane, 2,2-bis(4-hydroxyphenyl)-4-methylpentane, 2,2-bis(4-hydroxyphenyl)heptane, 4,4-bis(4-hydroxyphenyl)heptane, 2,2-bis(4-hydroxyphenyl)tridecane, 2,2-bis(4-hydroxyphenyl)octane, 2,2-bis(3-methyl-4-hydroxyphenyl)propane, 2,2-bis(3-ethyl-4-hydroxyphenyl)propane, 2,2-bis(3-n-propyl-4-hydroxyphenyl)propane, 2,2-bis(3-isopropyl-4-hydroxyphenyl)propane, 2,2-bis(3-sec-butyl-4-hydroxyphenyl)propane, 2,2-bis(3-tert-butyl-4-hydroxyphenyl)propane, 2,2-bis(3-cyclohexyl-4-hydroxyphenyl)propane, 2,2-bis(3-allyl-4′-hydroxyphenyl)propane, 2,2-bis(3-methoxy-4-hydroxyphenyl)propane, 2,2-bis(3,5-dimethyl-4-hydroxyphenyl)propane, 2,2-bis(2,3,5,6-tetramethyl-4-hydroxyphenyl)propane, bis(4-hydroxyphenyl)cyanomethane, 1-cyano-3,3-bis(4-hydroxyphenyl)butane, 2,2-bis(4-hydroxyphenyl)hexafluoropropane, 1,1-bis(4-hydroxyphenyl)cyclopentane, 1,1-bis(4-hydroxyphenyl)cyclohexane, 1,1-bis(4-hydroxyphenyl)cycloheptane, 1,1-bis(3-methyl-4-hydroxyphenyl)cyclohexane, 1,1-bis(3,5-dimethyl-4-hydroxyphenyl)cyclohexane, 1,1-bis(3,5-dichloro-4-hydroxyphenyl)cyclohexane, 1, 1-bis(3-methyl-4-hydroxyphenyl)-4-methylcyclohexane, 1,1-bis(4-hydroxyphenyl)-3,3,5-trimethylcyclohexane, 2,2-bis(4-hydroxyphenyl)norbornane, 2,2-bis(4-hydroxyphenyl)adamantane, 4,4′-dihydroxydiphenyl ether, 4,4′-dihydroxy-3,3′-dimethyldiphenyl ether, ethylene glycol bis(4-hydroxyphenyl) ether, 4,4′-dihydroxydiphenyl sulfide, 3,3′-dimethyl-4,4′-dihydroxydiphenyl sulfide, 3,3′-dicyclohexyl-4,4′-dihydroxydiphenyl sulfide, 3,3′-diphenyl-4,4′-dihydroxydiphenyl sulfide, 4,4′-dihydroxydiphenyl sulfoxide, 3,3′-dimethyl-4,4′-dihydroxydiphenyl sulfoxide, 4,4′-dihydroxydiphenyl sulfone, 4,4′-dihydroxy-3,3′-dimethyldiphenyl sulfone, bis(4-hydroxyphenyl ketone, bis(4-hydroxy-3-methylphenyl) ketone, 7,7′-dihydroxy-3,3′,4,4′-tetrahydro-4,4,4′,4′-tetramethyl-2,2′-spirobi(2H-1-benzopyrane), trans-2,3-bis(4-hydroxyphenyl)-2-butene, 9,9-bis(4-hydroxyphenyl)fluorene, 3,3-bis(4-hydroxyphenyl)-2-butanone, 1,6-bis(4-hydroxyphenyl)-1,6-hexanedione, 4,4′-dihydroxybiphenyl, m-dihydroxyxylylene, p-dihydroxyxylylene, 1,4-bis(2-hydroxyethyl)benzene, 1,4-bis(3-hydroxypropyl)benzene, 1,4-bis(4-hydroxybutyl)benzene, 1,4-bis(5-hydroxypentyl)benzene, 1,4-bis(6-hydroxyhexyl)benzene, 2,2-bis[4-(2″-hydroxyethyloxy)phenyl]propane, hydroquinone, and resorcin.

Polyfunctional polyol compounds, for example, trihydroxynaphtalene, tetrahydroxynaphthalene, benzenetriol, biphenyltetraol, pyrogallol, (hydroxynaphthyl)pyrogallol, and trihydroxyphenanthrene.

### Polyester Polyol

Examples thereof include a compound obtained through condensation reaction of a polyol and a compound having plural carboxylic acids. In particular, the number average molecular weight thereof is preferably 400 to 2,000, more preferably 500 to 1,500, and most preferably 600 to 1,200. A compound having hydroxy groups only at both ends of the molecule (i.e., two hydroxy groups) corresponds to the bifunctional polyol (C12) constituting the urethane prepolymer (B12).

Examples of the polyol include ethylene glycol, 1,2-propanediol, 1,3-butanediol, 1,4-butanediol, 3-methyl-1,5-pentanediol, 1,6-hexanediol, 3,3′-dimethylolheptane, 1,4-cyclohexanedimethanol, neopentyl glycol, 3,3-bis(hydroxymethyl)heptane, diethylene glycol, dipropylene glycol, glycerin, and trimethylolpropane, which may be used alone or as a mixture of two or more kinds thereof. Examples of the compound having plural carboxylic acids include succinic acid, adipic acid, azelaic acid, sebacic acid, dodecanedicarboxylic acid, cyclopentanedicarboxylic acid, cyclohexanedicarboxylic acid, orthophthalic acid, isophthalic acid, terephthalic acid, and naphthalenedicarboxylic acid, which may be used alone or as a mixture of two or more kinds thereof.

The polyester polyol is available industrially or as a reagent, and examples of the commercially available product thereof include “Polyite (registered trademark)” series, available from DIC Corporation, “Nippolan (registered trademark)” series, available from Nippon Polyurethane Industry Co., Ltd., “Maximol (registered trademark)” series, available from Kawasaki Kasei Chemicals Ltd., and “Kuraray Polyol (registered trademark)” series, available from Kuraray Co., Ltd.

### Polyether Polyol

Examples thereof include a compound obtained through ring-opening polymerization of an alkylene oxide or reaction of a compound having two or more active hydrogen-containing groups in the molecule and an alkylene oxide, and a modified product thereof. In particular, the number average molecular weight thereof is preferably 400 to 2,000, more preferably 500 to 1,500, and most preferably 600 to 1,200. A compound having hydroxy groups only at both ends of the molecule (i.e., two hydroxy groups) corresponds to the bifunctional polyol (C12) constituting the urethane prepolymer (B12).

Examples of the polyether polyol include a polymer polyol, a urethane-modified polyether polyol, and a polyether ester copolymer polyol, and examples of the compound having two or more active hydrogen groups in the molecule include water and a polyol compound, such as glycol or glycerin, having one or more hydroxy group, such as ethylene glycol, propylene glycol, butanediol, glycerin, trimethylolpropane, hexanetriol, triethanolamine, diglycerin, pentaerythritol, trimethylolpropane, and hexanetriol, which may be used alone or as a mixture of two or more kinds thereof.

Examples of the alkylene oxide include ethylene oxide, propylene oxide, and a cyclic ether compound, such as tetrahydrofuran, which may be used alone or as a mixture of two or more kinds thereof.

The polyether polyol is available industrially or as a reagent, and examples of the commercially available product thereof include “Excenol (registered trademark)” series and “Emulstar” (registered trademark)″ series, available from AGC Inc., and “Adeka Polyether (registered trademark)” series, available from Adeka Corporation.

### Polycaprolactone Polyol

Examples thereof include a compound obtained through ring-opening polymerization of s-caprolactone. In particular, the number average molecular weight thereof is preferably 400 to 2,000, more preferably 500 to 1,500, and most preferably 600 to 1,200. A compound having hydroxy groups only at both ends of the molecule (i.e., two hydroxy groups) corresponds to the bifunctional polyol (C12) constituting the urethane prepolymer (B12).

The polycaprolactone polyol is available industrially or as a reagent, and examples of the commercially available product thereof include “Placcel (registered trademark)” series, available from Daicel Corporation.

### Polycarbonate Polyol

Examples thereof include a compound obtained through phosgenation of one or more kind of a low molecular weight polyol, and a compound obtained through ester exchange using ethylene carbonate, diethyl carbonate, diphenyl carbonate, or the like. In particular, the number average molecular weight thereof is preferably 400 to 2,000, more preferably 500 to 1,500, and most preferably 600 to 1,200. A compound having hydroxy groups only at both ends of the molecule (i.e., two hydroxy groups) corresponds to the bifunctional polyol (C12) constituting the urethane prepolymer (B12).

Examples of the low molecular weight polyol include low molecular polyol compounds, such as ethylene glycol, 1,2-propanediol, 1,3-propanediol, 2-methyl-1,3-propanediol, 1,2-butanediol, 1,3-butanediol, 1,4-butanediol, 1,5-pentanediol, 1,6-hexanediol, 1,8-octanediol, 1,9-nonanediol, 1,10-decanediol, 3-methyl-1,5-pentanediol, 2-ethyl-4-butyl-1,3-propanediol, diethylene glycol, dipropylene glycol, neopentyl glycol, cyclohexane-1,4-diol, cyclohexane-1,4-dimethanol, dimer acid diol, an ethylene oxide or propylene oxide adduct of bisphenol A, bis(β-hydroxyethyl)benzene, xylylene glycol, glycerin, trimethylolpropane, and pentaerythritol.

### Polyacrylic Polyol

Examples thereof include a polyol compound obtained through polymerization of a (meth)acrylate ester and a vinyl monomer. A compound having hydroxy groups only at both ends of the molecule (i.e., two hydroxy groups) corresponds to the bifunctional polyol (C12) constituting the urethane prepolymer (B12).

### Castor Oil Based Polyol

Examples of the castor oil based polyol include a polyol compound obtained from castor oil, which is a natural oil, as a starting material. A compound having hydroxy groups only at both ends of the molecule (i.e., two hydroxy groups) corresponds to the component (C12) constituting the urethane prepolymer (B1).

The castor oil polyol is available industrially or as a reagent, and examples of the commercially available product thereof include “URIC (registered trademark)” series, available from Itoh Oil Chemicals Co., Ltd.

### Polyamine Compound (C2)

The polyamine compound (C2) used in the present invention may be any polyamine compound that has two or more primary or secondary amino groups in one molecule, with no limitation. The polyamine compound can be roughly classified into an aliphatic amine, an alicyclic amine, and an aromatic amine.

Specific examples of the polyamine compound (C2) include the following compounds.

### Aliphatic Amine

Bifunctional amine compounds (corresponding to the bifunctional amine compound (C22) constituting the urethane prepolymer (B12)), for example, ethylenediamine, hexamethylenediamine, nonamethylenediamine, undecanemethylenediamine, dodecamethylenediamine, m-xylenediamine, 1,3-propanediamine, and putrescine.

Polyfunctional amine compounds, for example, diethylenetriamine.

### Alicyclic Amine

Bifunctional amine compounds (corresponding to the bifunctional amine compound (C22) constituting the urethane prepolymer (B12)), for example, isophoronediamine and cyclohexyldiamine.

### Aromatic Amine

Bifunctional amine compounds (corresponding to the bifunctional amine compound (C22) constituting the urethane prepolymer (B12)), for example, 4,4′-methylenebis(o-chloroaniline) (MOCA), 2,6-dichloro-p-phenylenediamine, 4,4′-methylenebis(2,3-dichloroaniline), 4,4′-methylenebis(2-ethyl-6-methylaniline), 3,5-bis(methylthio)-2,4-toluenediamine, 3,5-bis(methylthio)-2,6-toluenediamine, 3,5-diethyltoluene-2,4-diamine, 3,5-diethyltoluene-2,6-diamine, trimethylene glycol-di-p-aminobenzoate, polytetramethylene glycol-di-p-aminobenzoate, 4,4′-diamino-3,3′,5,5′-tetraethyldiphenylmethane, 4,4′-diamino-3,3′-diisopropyl-5,5′-dimethyldiphenylmethane, 4,4′-diamino-3,3′,5,5′-tetraisopropyldiphenylmethane, 1,2-bis(2-aminophenylthio)ethane, 4,4′-diamino-3,3′-diethyl-5,5′-dimethyldiphenylmethane, N,N′-di-sec-butyl-4,4′-diaminodiphenylmethane, 3,3′-diethyl-4,4′-diaminodiphenylmethane, m-xylylenediamine, N,N′-di-sec-butyl-p-phenylenediamine, m-phenylenediamine, p-xylylenediamine, p-phenylenediamine, 3,3′-methylenebis(methyl-6-aminobenzoate), 2-methylpropyl 2,4-diamino-4-chlorobenzoate, isopropyl 2,4-diamino-4-chlorobenzoate, isopropyl 2,4-diamino-4-chlorophenylacetate, di(2-aminophenyl)thioethyl terephthalate, diphenylmethanediamine, tolylenediamine, and piperazine.

Polyfunctional amine compounds, for example, 1,3,5-benzenetriamine and melamine.

### Polythiol Component (C3)

The polythiol compound (C3) used in the present invention may be any polythiol compound that has two or more thiol groups in one molecule, with no limitation. For preferred specific examples of the polythiol, reference may be made to the compounds described in WO 2015/068798. Particularly preferred examples among these include the following compounds.

Tetraethylene glycol bis(3-mercaptopropionate), 1,4-butanediol bis(3-mercaptopropionate), 1,6-hexanediol bis(3-mercaptopropionate), and 1,4-bis(mercaptopropylthiomethyl)benzenol (corresponding to the bifunctional polythiol compound (32) constituting the urethane prepolymer (B12)).

Polyfunctional polythiol compounds, for example, trimethylolpropane tris(3-mercaptopropionate), pentaerythritol tetrakis(3-mercaptopropionate), dipentaerythritol hexakis(3-mercaptopropionate), 1,2-bis[(2-mercaptoethyl)thio]-3-mercaptopropane, 2,2-bis(mercaptomethyl)-1,4-butanedithiol, 2,5-bis(mercaptomethyl)-1,4-dithiane, 4-mercaptomethyl-1,8-dimercapto-3,6-dithiaoctane, 1,1,1,1-tetrakis(mercaptomethyl)methane, 1,1,3,3-tetrakis(mercaptomethylthio)propane, 1,1,2,2-tetrakis(mercaptomethylthio)ethane, 4,6-bis(mercaptomethylthio)-1,3-dithiane, and tris [(3-mercaptopropyonyloxy)ethyl]-isocyanurate.

### Compound Containing at Least Two Kinds of Active Hydrogen Groups (C4)

The compound containing at least two kinds of active hydrogen groups (C4) may be any compound that contains one or more of each of at least two kinds of active hydrogen groups selected from a hydroxy group, an amino group, and a thiol group, with no limitation.

### Hydroxy Group and Thiol Group-Containing Compound

The hydroxy group and thiol group-containing compound is a compound that contains a hydroxy group and a thiol group in one molecule. Specific examples thereof include the following compounds.

Bifunctional compounds containing two kinds of active hydrogen groups, for example, 2-mercaptoethanol, 1-hydroxy-4-mercaptocyclohexane, 2-mercaptohydroquinone, 4-mercaptophenol, 1-hydroxyethylthio-3-mercaptoethylthiobenzene, 4-hydroxy-4′-mercaptodiphenyl sulfone, 2-(2-mercaptoethylthio)ethanol, dihydroxyethyl sulfide mono(3-mercaptopropionate), and dimercaptoethane mono(salicylate).

Polyfunctional compounds containing two kinds of active hydrogen groups, for example, 3-mercapto-1,2-propanediol, glycerin di(mercaptoacetate), 2,4-dimercaptophenol, 1,3-dimercapto-2-propanol, 2,3-dimercapto-1-propanol, 1,2-dimercapto-1,3-butanediol, pentaerythritol tris(3-mercaptopropionate), pentaerythritol mono(3-mercaptopropionate), pentaerythritol bis(3-mercaptopropionate), pentaerythritol tris(thioglycolate), pentaerythritol pentakis(3-mercaptopropionate), hydroxymethyl-tris(mercaptoethylthiomethyl)methane, and hydroxyethylthiomethyl-tris(mercaptoethylthio)methane.

### Hydroxy Group and Amino Group-Containing Compound

The hydroxy group and amino group-containing compound is a compound that contains a hydroxy group and an amino group in one molecule. Specific examples thereof include the following compounds.

Bifunctional compounds containing two kinds of active hydrogen groups, for example, monoethanolamine, monopropanolamine, and N-methylethanolamine.

Polyfunctional compounds containing two kinds of active hydrogen groups, for example, diethanolamine, 2-(2-aminoethylamino)ethanol, 2-amino-2-hydroxymethylpropane-1,3-diol, 2-hydroxyethylethylenediamine, 2-hydroxyethylpropylenediamine, N,N-di-2-hydroxyethylethylenediamine, N,N-di-2-hydroxypropylethylenediamine, N,N-di-2-hydroxypropylpropylenediamine, N,N-di-2-hydroxyethylethylenediamine, N,N-di-2-hydroxypropylethylenediamine, and N,N-di-2-hydroxypropylpropylenediamine.

### Polyrotaxane Containing at Least Two Active Hydrogen Groups (C5)

A polyrotaxane is a composite of molecules having a structure in which an axial molecule is included in rings of plural ring molecules, and the ring molecules are interlocked to the axial molecule through steric hindrance of bulky groups bonded to both ends of the axial molecule, and may also be referred to as a supermolecule.

The polyrotaxane containing at least two active hydrogen groups (C5) used in the present invention is not particularly limited, as far as the polyrotaxane has at least two active hydrogen groups, and examples thereof include polyrotaxanes described in WO 2018/092826.

Examples of the axial molecule in the present invention include polyethylene glycol, polyisoprene, polyisobutylene, polybutadiene, polypropylene glycol, polytetrahydrofuran, polydimethylsiloxane, polyethylene, polypropylene, polyvinyl alcohol, and polyvinyl methyl ether, and polyethylene glycol is preferably used.

The molecular weight of the axial molecule is not particularly limited, and a too large molecular weight tends to increase the viscosity, whereas a too small molecular weight tends to decrease the mobility of the ring molecules. From the standpoint, the weight average molecular weight Mw of the axial molecule is preferably in a range of 400 to 100,000, particularly 1,000 to 80,000, and particularly preferably 2,000 to 50,000.

The ring molecule has a ring having a size in which the axial molecule described above can be included, and the ring is preferably a cyclodextrin ring. The cyclodextrin ring includes an α-type (inner diameter of ring: 0.45 to 0.6 nm), a β-type (inner diameter of ring: 0.6 to 0.8 nm), and a γ-type (inner diameter of ring: 0.8 to 0.95 nm), and an α-cyclodextrin ring is most preferred.

In the polyrotaxane, one axial molecule is included in plural ring molecules, and assuming that the maximum inclusion number of the ring molecules that can include one axial molecule is 1.0, the inclusion number of the ring molecules is preferably 0.8 or less at most. It is more preferred that one axial molecule is included in at least two ring molecules, and the inclusion number of the ring molecules is in a range of 0.5 or less at most.

In the present invention, the polyrotaxane containing at least two active hydrogen groups (C5) is a polyrotaxane that contains least two active hydrogen groups in the molecule, and has a feature of having active hydrogen capable of being polymerized with the component (B). In particular, it is preferred that the ring molecules have at least two active hydrogen groups, and it is more preferred that a side chain is introduced to the ring molecule, and the side chain has an active hydrogen group.

Examples of the active hydrogen group include at least one kind of a group selected from a hydroxy group, a thiol group, and an amino group.

The method of introducing an active hydrogen group to the side chain is not particularly limited, and a method of introducing a target side chain by reacting an organic chain having an active hydrogen group with a functional group of the ring molecules through ring-opening polymerization, radical polymerization, cation polymerization, anion polymerization, or the like.

### Preferred Mixing Ratio

The content of the component (A) in the curable composition containing the component (A) and the component (B) of the present invention is preferably 2 to 50 parts by mass per 100 parts by mass in total of the component (A) and the component (B). In the case where the component (A) is contained in this proportion, a cured product exhibiting excellent characteristics can be obtained. In the case where the component (A) is applied to a polishing pad for CMP, excellent polishing characteristics can be exhibited.

In particular, the content of the component (A) is more preferably 3 to 40 parts by mass, and further preferably 4.5 to 30 parts by mass, per 100 parts by mass in total of the component (A) and the component (B).

In the case where the component (C) is further added to the curable composition, the content of the component (A) in the curable composition containing the component (A), the component (B), and the component (C) of the present invention is preferably 1 to 20 parts by mass per 100 parts by mass in total of the component (A), the component (B), and the component (C).

In particular, the content of the component (A) is more preferably 2 to 18 parts by mass, and further preferably 2 to 15 parts by mass, per 100 parts by mass in total of the component (A), the component (B), and the component (C).

In the case where the cured product is applied to a polishing pad for CMP, it is preferred that the component (C) contains at least one kind selected from the polyamine (C2) and the trifunctional or higher polyol (C1), and it is particularly preferred that both the polyamine (C2) and the trifunctional or higher polyol (C1) are contained. According to the configuration, excellent polishing characteristics can be exhibited.

### Other Components Mixed in Curable Composition

The curable composition of the present invention may contain a polymerization curing accelerator (D) (which may be hereinafter referred to as a component (D)) in addition to the component (A), the component (B), and the component (C) described above.

As for the component (D), in the case where the polyfunctional active hydrogen group-containing sulfonate quaternary ammonium salt (A) is reacted with the polyfunctional isocyanate compound having at least two isocyanate groups (B1) of the polyfunctional isothiocyanate compound having at least two isothiocyanate groups (B2), a reaction catalyst for urethane or urea (D1) and a condensation agent (D2) may be used as the polymerization curing accelerator, and in the case where the polyfunctional active hydrogen group-containing sulfonate quaternary ammonium salt (A) is reacted with the epoxy group-containing compound (B3) or the episulfide group-containing compound (B4), an epoxy curing agent (D3) and a cation polymerization catalyst (D4) for performing ring-opening polymerization of an epoxy group may be used as the polymerization curing accelerator.

For specific examples of the polymerization accelerators (D1) to (D4) that are preferably used in the present invention, reference may be made to the compounds described in WO 2015/068798.

One kind of the components (D) may be used alone, or two or more kinds thereof may be used in combination. The amount thereof used may be a so-called catalyst amount, and for example, may be in a range of 0.001 to 10 parts by mass, and particularly 0.01 to 5 parts by mass, per 100 parts by mass in total of the component (A), the component (B), and the component (C).

In the curable composition of the present invention, various known mixing components may be additionally used in such a range that does not impair the effects of the present invention. For example, abrasive grains, an antioxidant, an ultraviolet ray absorbent, an infrared ray absorbent, a coloration preventing agent, a fluorescent dye, a dye, a photochromic compound, a pigment, a perfume, a surfactant, a flame retardant, a plasticizer, a filler, an antistatic agent, a foam stabilizer, a solvent, a leveling agent, and other additives may be added. These additives may be used alone or as a combination of two or more kinds thereof. These additives can be contained in the cured product by adding to the curable composition and then polymerizing the curable composition. Specific examples of the abrasive grains include particles formed of a material selected from cerium oxide, silicon oxide, alumina, silicon carbide, zirconia, iron oxide, manganese dioxide, titanium oxide, and diamond, and two or more kinds of particles selected from these materials.

The method of curing the curable composition of the present invention may be a known method. For example, the conditions described in WO 2018/092826 and WO 2019/198675 may be employed.

The cured product obtained by curing the curable composition of the present invention may have fine pores provided in the cured product depending on the purpose thereof. As the purpose of this type, a polishing pad for CMP has been known. The method of providing fine pores for applying to a polishing pad for CMP may be any known foaming method or the like, with no limitation. Examples of the method include a method of dispersing and curing a volatile foaming agent, such as a low boiling point hydrocarbon, or fine hollow particles (microballoons), a method of mixing a thermally expandable fine particles and then heating to foam the fine particles, and a mechanical froth foaming method of blowing air or an inert gas, such as nitrogen, during mixing. In the case where the curable composition capable of forming a urethane bond is used in the cured product of the present invention, a foaming agent foaming method of adding water or the like may also be used. Among these, fine hollow particles, which are preferably used in the case where the cured product is a foamed article, are preferred.

The fine hollow particles (E) (which may be hereinafter referred to as a component (E)) may be a known material with no limitation. Specific examples thereof used include particles having an outer shell formed of a vinylidene chloride resin, a (meth)acrylate resin, an acrylonitrile-vinylidene chloride copolymer, an epoxy resin, a phenol resin, a melamine resin, a urethane resin, or the like. Among these, the component (E) is preferably hollow particles having an outer shell formed of a urethane based resin and a hollow part surrounded by the outer shell. The urethane based resin is a resin having a urethane bond and/or a urea bond. The use of the hollow particles can easily provide a uniform foamed article efficiently. Furthermore, the use of the hollow particles prevents defects, such as scratches, from occurring, and also reduces the hysteresis loss.

The average particle diameter of the component (E) is not particularly limited, and is preferably in the following range. Specifically, the average particle diameter thereof is preferably 1 µm to 500 µm, more preferably 5 µm to 200 µm, and most preferably 10 to 100 µm.

The density of the component (E) is also not particularly limited, and is preferably in the following range. Specifically, the density thereof is preferably 0.01 g/cm³ to 0.5 g/cm³, and more preferably 0.02 g/cm³ to 0.3 g/cm³. The density herein is the density of the component (E) after expanding. The component (E) that is unexpanded particles in the stage of mixing with the curable composition and is expanded by the heat for curing preferably has the aforementioned density after expanding.

The amount of the component (E) mixed may be appropriately determined depending on the target purpose. In the case where the resulting cured product is used as a material of a polishing pad for CMP, the mixing amount is preferably as follows. Specifically, the amount of the component (E) is preferably 0.1 to 20 parts by mass, more preferably 0.2 to 10 parts by mass, and further preferably 0.5 to 8 parts by mass, per 100 parts by mass in total of the component (A), the component (B), and the component (C).

### Cured Product: Polishing Pad for CMP

In the present invention, in the case where the component (B) is selected from the component (B1) or the component (B2), the cured product is a cured product formed of a urethane based resin. In the case where the cured product is used as a polishing pad for CMP, the component (B) is preferably selected from the component (B1). In the present invention, the urethane based resin means a resin having at least one or more kind of a bond selected from a urethane bond, a urea bond, a thiourethane bond, and a thiourea bond.

The cured product obtained from the curable composition of the present invention particularly in the case where the cured product is a urethane based resin is preferably used as a material of a polishing pad for CMP due to the excellent mechanical characteristics thereof. The urethane based resin may have an optional appropriate hardness. The hardness can be measured according to the Shore method, and can be measured, for example, according to JIS K6253 (hardness test). The urethane based resin preferably has a Shore hardness of 20A to 90D. In the present invention, the Shore hardness of the urethane based resin used as a material of the polishing pad is preferably 30A to 70D, and more preferably 40A to 60D (“A” shows a hardness according to the Shore A scale, and “D” shows a hardness according to the Shore D scale). The hardness can be optionally set depending on necessity by changing the mixing composition and the mixing amounts.

The abrasion resistance of the polishing pad for CMP of the present invention is preferably 60 mg or less, and more preferably 50 mg or less, in the Taber abrasion test. With a smaller Taber abrasion amount, excellent abrasion resistance can be exhibited in using as a polishing pad for CMP. The detailed method for performing the Taber abrasion test may be the method shown in the examples described later.

The urethane based resin preferably has a compression rate in a certain range from the standpoint of exhibiting the flatness of the article to be polished. The compression rate can be measured by a method according to JIS L1096. The compression rate of the urethane based resin is preferably 0.5% to 50%. Within the range, excellent flatness of the article to be polished can be exhibited.

In the case where the polishing pad is constituted by multiple layers, the urethane based resin may be used as the material of the polishing pad in any of the layers. For example, in the case where the polishing pad is constituted by two layers, the configuration thereof preferably includes the first layer having a polishing surface in contact with the article to be polished in polishing, and the second layer in contact with the first layer on the surface opposite to the polishing surface of the first layer. In this case, the second layer and the first layer may be different in hardness or elastic modulus, and thereby the characteristics of the polishing pad for CMP can be regulated. For example, the hardness of the first layer and the hardness of the second layer may be allowed to differ from each other, and thereby the polishability of the polished article can be regulated.

In the present invention, in particular, both the first and second layers are preferably formed of the urethane based resin described above.

The urethane based resin that is used as a material for a polishing pad for CMP may contain abrasive grains to be a fixed abrasive grain urethane based resin. Examples of the abrasive grains include particles formed of a material selected from cerium oxide, silicon oxide, alumina, silicon carbide, zirconia, iron oxide, manganese dioxide, titanium oxide, and diamond, and two or more kinds of particles selected from these materials. The method of containing the abrasive grains is not particularly limited, and for example, the abrasive grains can be contained in the urethane based resin by dispersing the abrasive grains in the curable composition and then curing the curable composition.

In the present invention, the polishing pad for CMP is not particularly limited, and a groove structure may be formed on the surface thereof. The groove structure preferably has a shape capable of retaining and renewing the slurry in polishing an article to be polished. Specific examples thereof include X (stripe) grooves, XY lattice grooves, concentric grooves, through holes, blind holes, polygonal columns, circular columns, spiral grooves, eccentric grooves, radial grooves, and combinations of these grooves.

The method of producing the grooves is not particularly limited. Examples thereof include a method of mechanically cutting with a tool having a prescribed size, such as a cutting bite, a method of casting the resin in a metal mold having a prescribed surface shape and then curing the resin, a method of pressing the resin with a pressing plate having a prescribed surface shape, a method using photolithography, a method using a printing method, and a method using laser light, such as carbon dioxide laser.

In the present invention, a polishing pad for CMP in the form of nonwoven fabric can be formed by coating or impregnating a nonwoven fabric with the curable composition, followed by curing.

The purpose of the cured product of the present invention includes a buffer material, a damping material, an acoustic absorbing material, and the like, in addition to the polishing pad for CMP. In the present invention, furthermore, the polishing pad for CMP in the form of nonwoven fabric formed by coating or impregnating a nonwoven fabric with the cured product composition, followed by curing may be applied not only to the polishing pad for CMP in the form of nonwoven fabric, but also to such purposes as a buffer material, a damping material, an acoustic absorbing material, and the like.

### EXAMPLES

The present invention will be described in detail with reference to examples and comparative examples below, but the present invention is not limited to the examples. The components and the evaluation methods used in the examples and the comparative examples below were as follows.

The resulting compound was dissolved in deuterated water, and then identified with an ¹H nuclear magnetic resonance apparatus (JNM-ECA400II, available from JEOL, Ltd.).

### Production Method of Polyfunctional Active Hydrogen Group-Containing Sulfonate Quaternary Ammonium Salt

### Example 1: Sulfonate Imidazolium Salt 1

### 1) Preparation of 1-Ethyl-3-Methylimidazolium Hydroxide

25 g of 1-ethyl-3-methylimidazolium chloride was dissolved in ion exchanged water, and then charged in a column filled with Amberlite IRA 400J as an OH type anion exchange resin, so as to exchange chloride ion to hydroxide ion. The commercially available IRA 400J was in the C1 type, and therefore used after converting to the OH type through ion exchange with an alkali. The resulting 1-ethyl-3-methylimidazolium salt was confirmed by ion chromatography that 99% or more of chloride ion therein was exchanged to hydroxide ion.

### 2) Preparation of Sulfonate Imidazolium Salt 1

36.36 g of N,N-bis(2-hydroxyethyl)-2-aminoethanesulfonic acid was added to the aqueous solution of 1-ethyl-3-methylimidazolium hydroxide prepared above, and after agitating for 1 hour, water was distilled off therefrom to provide a sulfonate imidazolium salt 1.

The proton nuclear magnetic resonance spectrum measured for the resulting sulfonate imidazolium salt 1 showed a 3H peak derived from 1-ethyl-3-methylimidazolium hydroxide around 1.0 to 2.0 ppm, a 12H peak derived from N,N-bis(2-hydroxyethyl)-2-aminoethanesulfonic acid around 2.0 to 3.8 ppm, a 5H peak derived from 1-ethyl-3-methylimidazolium hydroxide around 3.8 to 4.5 ppm, a 2H peak derived from 1-ethyl-3-methylimidazolium hydroxide around 7.0 to 8.0 ppm, and a 1H peak derived from 1-ethyl-3-methylimidazolium hydroxide around 8.0 to 9.0 ppm.

The structural formula of the resulting sulfonate imidazolium salt 1 is shown by the following formula (8).

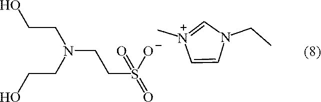

The sulfonate imidazolium salt 1 shown by the formula (8) was a polyfunctional active hydrogen group-containing sulfonate quaternary ammonium salt that was in a liquid state at room temperature.

### Example 2: Sulfonate Imidazolium Salt 2

A sulfonate imidazolium salt 2 was obtained in the same manner as in Example 1 except that 34.57 g of 1-hexyl-3-methylimidazolium chloride was used instead of 25 g of 1-ethyl-3-methylimidazolium chloride used in the item (1-1).

The proton nuclear magnetic resonance spectrum measured for the resulting sulfonate imidazolium salt 2 showed an 11H peak derived from 1-hexyl-3-methylimidazolium hydroxide around 1.0 to 2.0 ppm, a 12H peak derived from N,N-bis(2-hydroxyethyl)-2-aminoethanesulfonic acid around 2.0 to 3.8 ppm, a 5H peak derived from 1-hexyl-3-methylimidazolium hydroxide around 3.8 to 4.5 ppm, a 2H peak derived from 1-hexyl-3-methylimidazolium hydroxide around 7.0 to 8.0 ppm, and a 1H peak derived from 1-hexyl-3-methylimidazolium hydroxide around 8.0 to 9.0 ppm. The structural formula of the resulting sulfonate imidazolium salt 2 is shown by the following formula (9).

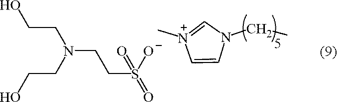

The sulfonate imidazolium salt 2 shown by the formula (9) was a polyfunctional active hydrogen group-containing sulfonate quaternary ammonium salt that was in a liquid state at room temperature.

### Example 3: Sulfonate Imidazolium Salt 3

A sulfonate imidazolium salt 3 was obtained in the same manner as in Example 1 except that 44.13 g of 1-decyl-3-methylimidazolium chloride was used instead of 25 g of 1-ethyl-3-methylimidazolium chloride used in the item (1-1).

The proton nuclear magnetic resonance spectrum measured for the resulting sulfonate imidazolium salt 3 showed a 19H peak derived from 1-decyl-3-methylimidazolium hydroxide around 1.0 to 2.0 ppm, a 12H peak derived from N,N-bis(2-hydroxyethyl)-2-aminoethanesulfonic acid around 2.0 to 3.8 ppm, a 5H peak derived from 1-decyl-3-methylimidazolium hydroxide around 3.8 to 4.5 ppm, a 2H peak derived from 1-decyl-3-methylimidazolium hydroxide around 7.0 to 8.0 ppm, and a 1H peak derived from 1-decyl-3-methylimidazolium hydroxide around 8.0 to 9.0 ppm. The structural formula of the resulting sulfonate imidazolium salt 3 is shown by the following formula (10).

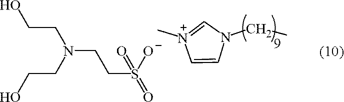

The sulfonate imidazolium salt 3 shown by the formula (10) was a polyfunctional active hydrogen group-containing sulfonate quaternary ammonium salt that was in a liquid state at room temperature.

### Example 4: Sulfonate Imidazolium Salt 4

A sulfonate imidazolium salt 4 was obtained in the same manner as in Example 1 except that 22.61 g of 1,3-dimethylimidazolium chloride was used instead of 25 g of 1-ethyl-3-methylimidazolium chloride used in the item (1-1).

The proton nuclear magnetic resonance spectrum measured for the resulting sulfonate imidazolium salt 4 showed a 12H peak derived from N,N-bis(2-hydroxyethyl)-2-aminoethanesulfonic acid around 2.0 to 3.8 ppm, a 6H peak derived from 1,3-dimethylimidazolium chloride around 3.8 to 4.5 ppm, a 2H peak derived from 1,3-dimethylimidazolium chloride around 7.0 to 8.0 ppm, and a 1H peak derived from 1,3-dimethylimidazolium chloride around 8.0 to 9.0 ppm. The structural formula of the resulting sulfonate imidazolium salt 4 is shown by the following formula (11).

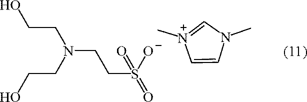

The sulfonate imidazolium salt 4 shown by the formula (11) was a polyfunctional active hydrogen group-containing sulfonate quaternary ammonium salt that was in a liquid state at room temperature.

### Example 21: Sulfonate Imidazolium Salt 5

A sulfonate imidazolium salt 5 was obtained in the same manner as in Example 1 except that 34.57 g of 1-hexyl-3-methylimidazolium chloride was used instead of 25 g of 1-ethyl-3-methylimidazolium chloride used in the item (1-1), and 41.48 g of N,N-bis(2-hydroxyethyl)-3-amino-2-hydroxypropanesulfonic acid was used instead of 36.36 g of N,N-bis(2-hydroxyethyl)-2-aminoethanesulfonic acid used therein. The proton nuclear magnetic resonance spectrum measured therefor showed an 11H peak derived from 1-hexyl-3-methylimidazolium hydroxide around 1.0 to 2.0 ppm, a 13H peak derived from N,N-bis(2-hydroxyethyl)-3-amino-2-hydroxpropanesulfonic acid around 2.0 to 4.0 ppm, a 5H peak derived from 1-ethyl-3-methylimidazolium hydroxide around 3.8 to 4.5 ppm, a 2H peak derived from 1-ethyl-3-methylimidazolium hydroxide around 7.0 to 8.0 ppm, and a 1H peak derived from 1-ethyl-3-methylimidazolium hydroxide around 8.0 to 9.0 ppm. The structural formula of the resulting sulfonate imidazolium salt 5 is shown by the following formula (14).

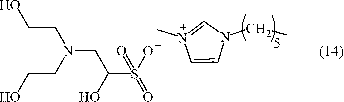

The sulfonate imidazolium salt 5 shown by the formula (14) was a polyfunctional active hydrogen group-containing sulfonate quaternary ammonium salt that was in a liquid state at room temperature.

### Comparative Example 1: Sulfonate Quaternary Ammonium Salt 1

14.13 g of N,N-bis(2-hydroxyethyl)-2-aminoethanesulfonic acid was added to 200 g of a 10% aqueous solution of hexadecyltrimethylammonium hydroxide, and after agitating for 1 hour, water was distilled off therefrom to provide a sulfonate quaternary ammonium salt 1. The structural formula thereof is shown by the following formula (12).

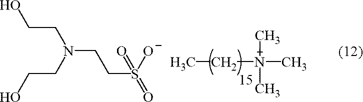

The sulfonate quaternary ammonium salt 1 shown by the formula (12) was in a solid state at room temperature.

### Comparative Example 2: Sulfonate Quaternary Ammonium Salt 2

A sulfonate quaternary ammonium salt 2 was obtained in the same manner as in Example 1 except that 48.40 g of 1-dodecylpyridinium chloride was used instead of 25 g of 1-ethyl-3-methylimidazolium chloride used in the item (1-1). The structural formula thereof is shown by the following formula (13).

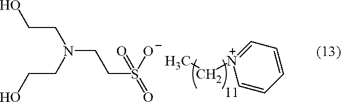

The sulfonate quaternary ammonium salt 2 shown by the formula (13) was in a waxy state at room temperature.

### Production Method of Cured Product

(B) Polymerizable monomer (B) having polymerizable functional group capable of being polymerized with polyfunctional active hydrogen group-containing sulfonate quaternary ammonium salt
Component (B1): Polyfunctional isocyanate compound having at least two isocyanate groups
Component (B12): Urethane prepolymer
Pre-1: Isocyanate-terminated urethane prepolymer having iso(thio)cyanate equivalent of 905

### Production Method of Pre-1

In a flask equipped with a nitrogen introducing tube, a thermometer, and an agitator, 50 g of 2,4-tolylenediisocyanate, 90 g of polyoxytetramethylene glycol (number average molecular weight: 1,000), and 12 g of diethylene glycol were reacted at 80° C. for 6 hours in a nitrogen atmosphere, so as to provide an isocyanate-terminated urethane prepolymer (Pre-1) having an isocyanate equivalent of 905.

(C) Active hydrogen-containing compound (C) other than polyfunctional active hydrogen group-containing sulfonate quaternary ammonium salt
Component (C1): Polyol
Poly #10: Polycastor #10, available from Itoh Oil Chemicals Co., Ltd., castor oil based polyol having 2.8 mmol/g of active hydrogen group per weight and pentafunctionality to hexafunctionality of hydroxy groups
Component (C2): Polyamine
MOCA: 4,4′-Methylenebis(o-chloroaniline)
Hartcure 30: Diamine, available from Kumiai Chemical Industry Co., Ltd., mixture of 3,5-diethyltoluene-2,4-diamine and 3,5-diethyltoluene-2,6-diamine.

### (E) Fine Hollow Particles

Fine hollow particles 1: Hollow microcapsules 920-40 having particle diameter of 40 µm and density of 0.03 g/cm³ (available from Nippon Ferrite Co., Ltd.)
Fine hollow particles 2: Hollow urethane resin microballoons having particle diameter of 30 µm and density of 0.13 g/cm³

### Production Method of Fine Hollow Particles 2

1,000 g of toluene was added to 650 g of polytetramethylene glycol (number average molecular weight: 2,000), to which 142 g of isophorone diisocyanate was further added, and the reaction was performed at 120° C. for 5 hours under refluxing toluene, followed by cooling to room temperature. 25 g of hexamethylenediamine and 20 g of diethylenetriamine were added to the reaction mixture and the reaction was performed at 60° C. for 5 hours, and then toluene was distilled off under reduced pressure, so as to provide a polyurethane resin having hydroxy groups at both ends thereof containing urethane and urea bonds. 400 g of the resulting resin, 12 g of iron oxide, 62 g of n-hexane, and 380 g of ethyl acetate were mixed, and the mixture was added in a dropwise manner and dispersed in 2,000 g of 0.5% aqueous solution of polyvinyl alcohol having been produced in advance. The resulting resin was taken out from water by filtering with filter paper, and dried at 40° C. with a circulation dryer. The spherical articles were pulverized and classified with a sonic classifier, so as to provide fine hollow particles 2.

### Example 5

### Production Method of Cured Product Using Sulfonate Imidazolium Salt 2

3.5 parts by mass of the sulfonate imidazolium salt 2 produced above and 9.7 parts by mass of 4,4′-methylenebis(o-chloroaniline) (MOCA) as the component (C) were mixed at 120° C. to form a uniform solution, which was then sufficiently deaerated to prepare a liquid A. 86.8 parts by mass of Pre-1 as the component (B) prepared above having been heated to 70° C. separately was added, and the mixture was agitated with a planetary centrifugal mixer to prepare a uniform curable composition. The curable composition was charged in a metal mold and cured at 100° C. for 15 hours to provide a cured product.

The cured product obtained above had a Shore hardness of 55D and a water absorption rate of 2.5%. The evaluation methods are shown below.

### Evaluation Items

(1) The Shore hardness was measured according to JIS K6253 (hardness test) with a durometer, available from Kobunshi Keiki Co., Ltd.
(2) Measurement of water absorption rate: The water absorption rate was measured by the cured product immersed in distilled water at 23° C. for 24 hours and the cured product immersed therein for 200 hours. The water absorption rate (%) was calculated from the weights of the specimens before and after immersion. Water absorption rate (%) = [(weight after immersion - weight before immersion) / weight before immersion] × 100
(3) Abrasion resistance: The Taber abrasion was measured with an apparatus type 5130, available from Taber Industries. The abrasion amount was measured by performing a Taber abrasion test at a load of 1 kg, a rotation speed of 60 rpm, and a rotation number of 1,000, with a wear ring H-18.

### Examples 6 to 11 and 15 to 17 and Comparative Example 3

Cured products were produced and evaluated in the same manner as in Example 1 except that the curable compositions having the compositions shown in Table 1 were used. The mixing ratios of the components and results are shown in Table 1.

### Example 12

### Production Method of Cured Product Using Sulfonate Imidazolium Salt 2

3.5 parts by mass of the sulfonate imidazolium salt 2 produced above and 8 parts by mass of Hartcure as the component (C) were mixed at room temperature to form a uniform solution, which was then sufficiently deaerated to prepare a liquid A. 88.5 parts by mass of Pre-1 as the component (B) prepared above having been heated to 70° C. separately was added, and the mixture was agitated with a planetary centrifugal mixer to prepare a uniform curable composition. The curable composition was charged in a metal mold and cured at 100° C. for 15 hours to provide a cured product.

The cured product obtained above had a Shore hardness of 54D and a water absorption rate of 2.5%. The evaluation methods are as described above.

TABLE 1

| | Curable composition | | | Evaluation result | | | |
|---|---|---|---|---|---|---|---|
| | Component (A) (part by mass) | Component (B) (part by mass) | Component (C) (part by mass) | Shore hardness | Water absorption rate 24 hours (%) | Water absorption rate 200 hours (%) | Abrasion resistance (mg) |
| Example 5 | sulfonate imidazolium salt 2 (3.5) | Pre-1 (86.8) | MOCA (9.7) | 55D | 2.5 | 4.5 | 30 |
| Example 6 | sulfonate imidazolium salt 2 (0.8) | Pre-1 (87.5) | MOCA (11.7) | 55D | 1.9 | 3.4 | 40 |
| Example 7 | sulfonate imidazolium salt 2 (1.7) | Pre-1 (87.3) | MOCA (11) | 56D | 2.2 | 4 | 35 |
| Example 8 | sulfonate imidazolium salt 2 (6.9) | Pre-1 (85.9) | MOCA (7.2) | 35D | 3.7 | 6.7 | 45 |
| Example 9 | sulfonate imidazolium salt 1 (3.0) | Pre-1 (87.3) | MOCA (9.7) | 53D | 3.5 | 8.7 | 34 |
| Example 10 | sulfonate imidazolium salt 3 (3.9) | Pre-1 (86.4) | MOCA (9.7) | 55D | 2.5 | 4.6 | 45 |
| Example 11 | sulfonate imidazolium salt 4 (2.7) | Pre-1 (87.9) | MOCA (9.4) | 54D | 3.5 | 7.8 | 30 |
| Example 12 | sulfonate imidazolium salt 2 (3.5) | Pre-1 (88.5) | Hartcure 30 (8.0) | 54D | 2.5 | 4.5 | 33 |
| Example 15 | sulfonate imidazolium salt 2 (3.0) | Pre-1 (77.4) | Poly #10 (22.6) | 25D | 2.5 | 4.5 | 42 |
| Example 16 | sulfonate imidazolium salt 2 (3.3) | Pre-1 (81.7) | MOCA (5.7) Poly #10 (9.3) | 30D | 2.5 | 4.6 | 30 |
| Example 17 | sulfonate imidazolium salt 5 (2.5) | Pre-1 (87.7) | MOCA (9.8) | 56D | 2.4 | 4.3 | 30 |
| Comparative Example 3 | - | Pre-1 (88.7) | MOCA (12.3) | 55D | 1.5 | 3.2 | 40 |

### Example 13

### Production Method of Polishing Pad for CMP Using Sulfonate Imidazolium Salt 2

3.5 parts by mass of the sulfonate imidazolium salt 2 produced above and 9.7 parts by mass of 4,4′-methylenebis(o-chloroaniline) (MOCA) as the component (C) were mixed at 120° C. to form a uniform solution, which was then sufficiently deaerated to prepare a liquid A. Separately, the hollow particles 1 (0.8 part by mass) as the component (E) were added to 86.8 parts by mass of Pre-1 as the component (B) prepared above having been heated to 70° C., and the mixture was agitated with a planetary centrifugal mixer to prepare a liquid B as a uniform solution. The liquid A was added to the liquid B prepared above and uniformly mixed to form a curable composition. The curable composition was charged in a metal mold and cured at 100° C. for 15 hours. After completing the polymerization, the urethane resin was taken out from the mold to provide a cured product.

The resulting cured product was sliced to provide a urethane resin having a thickness of 1 mm. Spiral grooves were formed on the surface of the urethane resin, and a double-sided adhesive tape was adhered to the back surface thereof, so as to provide a polishing pad for CMP formed of a urethane resin having a diameter of 500 mm and a thickness of 1 mm. The mixing amounts are shown in Table 2.

The polishing pad for CMP formed of the urethane resin obtained above had a density of 0.8 g/cm³ and a polishing rate of 2.2 µm/hr, and the surface roughness after polishing of the wafer as the article to be polished was 0.28 nm.

### Evaluation Methods

### Density

The density (g/cm³) was measured with DSG-1, available from Toyo Seiki Seisaku-sho, Ltd.

### Polishing Rate

The polishing rate in polishing under the following condition was measured. The polishing rate was an average value of 10 plies of 2-inch sapphire wafers.

Polishing pad for CMP: Pad having diameter of 500 mm and thickness of 1 mm having concentric grooves formed on surface
Slurry: Fujimi Compol 80 stock liquid
Pressure: 4 psi
Rotation number: 45 rpm
Time: 1 hour
(6) Surface roughness (Ra)

The surfaces of the 10 plies of 2-inch sapphire wafers polished under the condition shown in the item (5) above were measured for the surface roughness (Ra) with Nano Search Microscope SFT-4500 (available from Shimadzu Corporation). The surface roughness was an average value of the 10 plies of 2-inch sapphire wafers.

### Examples 14 and 18 to 20 and Comparative Example 4

Polishing pads for CMP formed of a urethane resin were produced and evaluated in the same manner as in Example 13 except that the compositions shown in Table 2 were used. The results are shown in Table 2.

TABLE 2

| | Curable composition | | | | Evaluation result | | |
|---|---|---|---|---|---|---|---|
| | Component (A) (part by mass) | Component (B) (part by mass) | Component (C) (part by mass) | Component (E) (part by mass) | Density | Polishing rate (µm/hr) | Wafer surface roughness Ra (nm) |
| Example 13 | sulfonate imidazolium salt 2 (3.5) | Pre-1 (86.8) | MOCA (9.7) | fine hollow particles 1 (0.8) | 0.8 | 2.2 | 0.28 |
| Example 14 | sulfonate imidazolium salt 2 (3.5) | Pre-1 (86.8) | MOCA (9.7) | fine hollow particles 2 (3.3) | 0.85 | 2.5 | 0.24 |
| Example 18 | sulfonate imidazolium salt 2 (3.0) | Pre-1 (74.4) | Poly #10 (22.6) | fine hollow particles 1 (0.8) | 0.8 | 2.2 | 0.29 |
| Example 19 | sulfonate imidazolium salt 2 (3.3) | Pre-1 (81.7) | MOCA (5.7) Poly #10 (9.3) | fine hollow particles 1 (0.8) | 0.8 | 2.8 | 0.27 |
| Example 20 | sulfonate imidazolium salt 5 (2.5) | Pre-1 (87.7) | MOCA (9.8) | fine hollow particles 1 (0.8) | 0.8 | 2.3 | 0.28 |
| Comparative Example 4 | - | Pre-1 (88.7) | MOCA (12.3) | fine hollow particles 1 (0.8) | 0.8 | 1 | 0.32 |

1. A polyfunctional active hydrogen group-containing sulfonate quaternary ammonium salt having at least two active hydrogen groups in a molecule and an imidazole skeleton as a quaternary ammonium salt skeleton.

2. The polyfunctional active hydrogen group-containing sulfonate quaternary ammonium salt according to claim 1, being in a liquid state at room temperature.

3. The polyfunctional active hydrogen group-containing sulfonate quaternary ammonium salt according to claim 1, represented by the following formula (1): 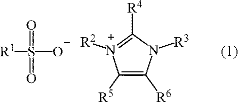
wherein in the formula,
R¹ represents an organic group having at least two active hydrogen groups;
R² and R³ each independently represent
a linear alkyl group having 1 to 20 carbon atoms, a linear alkenyl group having 1 to 20 carbon atoms, a branched alkyl group having 3 to 20 carbon atoms, or a branched alkenyl group having 3 to 20 carbon atoms,
in which a part of the linear alkyl group, the linear alkenyl group, the branched alkyl group, or the branched alkenyl group may be substituted by an —O— bond, a —CO— bond, a —COO—bond, an —NH— bond, an —SO— bond, or an —SiO— bond,
a part of the hydrogen atoms of the linear alkyl group, the linear alkenyl group, the branched alkyl group, or the branched alkenyl group may be substituted by at least one kind selected from the group consisting of a hydroxy group and a phenyl group, and
the groups represented by R² and R³ may be the same as or different from each other;
R⁴ represents a methyl group or a hydrogen atom; and
R⁵ and R⁶ each independently represent
a hydrogen atom,
a linear alkyl group having 1 to 20 carbon atoms, a linear alkenyl group having 1 to 20 carbon atoms, a branched alkyl group having 3 to 20 carbon atoms, or a branched alkenyl group having 3 to 20 carbon atoms,
in which a part of the linear alkyl group, the linear alkenyl group, the branched alkyl group, or the branched alkenyl group may be substituted by an —O— bond, a —CO— bond, a —COO—bond, an —NH— bond, an —SO— bond, or an —SiO— bond,
a part of the hydrogen atoms of the linear alkyl group, the linear alkenyl group, the branched alkyl group, or the branched alkenyl group may be substituted by at least one kind selected from the group consisting of a hydroxy group and a phenyl group,
the groups represented by R⁵ and R⁶ may be the same as or different from each other, and R⁵ and R⁶ and the carbon atoms bonded thereto may form an aliphatic hydrocarbon ring having 3 to 20 ring carbon atoms.

4. The polyfunctional active hydrogen group-containing sulfonate quaternary ammonium salt according to claim 3, represented by the following formula (2): 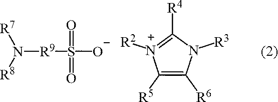
wherein in the formula,
R², R³, R⁴, R⁵, and R⁶ have the same meanings as in the formula (1);
R⁷ and R⁸ each independently represent
a hydrogen atom,
a linear alkyl group having 2 to 20 carbon atoms, at least one hydrogen atom of which is substituted by an active hydrogen group, or
a branched alkyl group having 3 to 20 carbon atoms, at least one hydrogen atom of which is substituted by an active hydrogen group,
in which a part of the linear alkyl group or the branched alkyl group may be substituted by an —O— bond, a —CO— bond, a —COO— bond, or an —NH— bond,
a part of the hydrogen atoms other than the active hydrogen group of the linear alkyl group or the branched alkyl group may be substituted by a phenyl group, and
the groups represented by R⁷ and R⁸ may be the same as or different from each other, but do not represent hydrogen atoms simultaneously; and
R⁹ represents
a linear alkylene group having 1 to 20 carbon atoms or
a branched alkylene group having 3 to 20 carbon atoms,
in which a part of the linear alkylene group or the branched alkylene group may be substituted by an —O— bond, a —CO— bond, a —COO— bond, or an —NH— bond, and
a part of the hydrogen atoms of the linear alkylene group or the branched alkylene group may be substituted by at least one kind selected from the group consisting of an active hydrogen group and a phenyl group,
provided that R⁷, R⁸, and R⁹ have at least two active hydrogen groups in total.

5. The polyfunctional active hydrogen group-containing sulfonate quaternary ammonium salt according to claim 1, wherein the active hydrogen group is at least one kind of a group selected from the group consisting of a hydroxy group and an amino group.

6. A curable composition comprising the polyfunctional active hydrogen group-containing sulfonate quaternary ammonium salt (A) according claim 1, and a polymerizable monomer (B) having a polymerizable functional group capable of being polymerized with the polyfunctional active hydrogen group-containing sulfonate quaternary ammonium salt (A).

7. The curable composition according to claim 6, wherein the polymerizable monomer (B) having a polymerizable functional group capable of being polymerized with the polyfunctional active hydrogen group-containing sulfonate quaternary ammonium salt (A) is a polyfunctional isocyanate compound having at least two isocyanate groups.

8. A cured product obtained by curing the curable composition according to claim 6.

9. A polishing pad for CMP comprising the cured product according to claim 8.
